(19) United States
(12) Patent Application Publication
(10) Pub. No.: US 2015/0306002 A1
(43) Pub. Date: Oct. 29, 2015

(54) 3-HYDROXY-4-OXO-4H-PYRAN- OR 3-HYDROXY-4-OXO-1,4-DIHYDROPYRIDINE DERIVATIVES AS PROTEIN-ADHESIVE ACTIVE SUBSTANCES

(71) Applicant: MERCK PATENT GMBH, Darmstadt (DE)

(72) Inventors: Thomas RUDOLPH, Darmstadt (DE); Philipp BUEHLE, Zwingenberg (DE)

(73) Assignee: Merck Patent GmbH, Darmstadt (DE)

(21) Appl. No.: 14/650,695

(22) PCT Filed: Nov. 14, 2013
(86) PCT No.: PCT/EP2013/003428

§ 371 Date: Jun. 9, 2015

(30) Foreign Application Priority Data
Dec. 13, 2012 (EP) 12008308.4

(51) Int. Cl. A61K 8/49; A61Q 17/04; A61K 8/64; C07D 309/40; C07D 405/12; C07D 213/69; A61Q 5/00; A61Q 19/08

(52) U.S. Cl. CPC A61K 8/498; A61Q 5/00; A61Q 17/04; A61Q 19/08; A61K 8/496; A61K 8/4926; C07D 309/40; C07D 405/12; C07D 213/69; A61K 8/64

(57) ABSTRACT

The invention relates to the use of 4-oxo-4H-pyran or 4-oxo-1,4-dihydropyridine derivatives as protein-adhesive active compounds, in particular as skin- or hair-adhesive and/or skin- or hair-binding UV absorbers, and for the protection of the skin, of the hair or of the nails against damage caused by ultraviolet radiation, to specific 4-oxo-4H-pyran or 4-oxo-1,4-dihydropyridine derivatives, to the preparation thereof and to preparations comprising same. The invention furthermore relates to the use of the structural unit 4-oxo-4H-pyran-3-O-yl or 4-oxo-1,4-dihydropyridin-3-O-yl as linker for the functionalisation of protein-containing matrices.

The invention relates to the use of 4-oxo-4H-pyran or 4-oxo-1,4-dihydropyridine derivatives as protein-adhesive active compounds, in particular as skin- or hair-adhesive and/or skin- or hair-binding UV absorbers, and for protection of the skin, of the hair or of the nails against damage caused by ultraviolet radiation, to specific 4-oxo-4H-pyran or 4-oxo-1,4-dihydropyridine derivatives, to the preparation thereof and to preparations comprising same. The invention furthermore relates to the use of the structural unit 4-oxo-4H-pyran-3-O-yl or 4-oxo-1,4-dihydropyridin-3-O-yl as linker for the functionalisation of protein-containing matrices.

A preferred area of application of the 4-oxo-4H-pyran or 4-oxo-1,4-dihydropyridine derivatives of the formula I, as described below, is UV protection or in other words protection of the skin, of the hair and of the nails, the so-called protein-containing matrices, against damage which can be caused by ultraviolet radiation or in other words for protection against UV-induced damage of the skin, of the hair and of the nails. Damage which can be caused by ultraviolet radiation is, for example, sun allergy, the generation of erythemas or sunburn, the formation of skin cancer and a weakening of the immune system, in particular of the immune system of the skin. The human skin is naturally also subject to certain ageing processes, some of which are attributable to intrinsic processes (chronoageing) and some of which are attributable to exogenous factors (environmental, for example photoageing). Photoageing is accompanied by a loss of the elasticity of the skin and pigment spots.

Ultraviolet radiation is, for example, ultraviolet solar radiation or sun-like light. Sun-like light is taken to mean radiation in a spectral composition analogous to solar radiation or sunlight. Sun-like light of this type can be generated, for example, by a solar simulator or used in solaria, where the spectral composition of the solaria radiation is not exactly equal to sunlight. Accordingly, sun-like light is also intended to be taken to mean light which is at least partly equal to the spectral composition of sunlight or which develops a sunlight-like effect.

The exogenous factors include, in particular, sunlight or artificial radiation sources having a comparable spectrum and compounds which may be formed by the radiation, such as undefined reactive photoproducts, which may also be free-radical or ionic.

A multiplicity of organic and inorganic UV filters and antioxidants is known which absorb UV radiation and are able to scavenge free radicals. They are thus capable of protecting the human skin. These compounds catalyse the transformation of UV light into heat.

Owing to poor skin adhesion, however, the duration of protection is limited, in particular since conventional UV absorbers or UV filters can be washed off very easily, for example by sweat or water.

WO 2006/018104, for example, discloses a strategy for derivatising UV absorbers, UV filters or self-tanner substances in such a way that they are able to bond covalently to the stratum corneum of the epidermis via a reactive moiety and thus functionalise the skin with the UV absorber, UV filter or self-tanner. For effective binding of proteins and amino acids of the outer skin layers, it is necessary for the corresponding UV absorbers, UV filters or self-tanners to have the highest possible reactivity of their moieties which are capable of binding.

Ascorbic acid derivatives, in particular of ascorbic acid derivatives which are substituted in the 6- and/or 5-position by UV chromophores, are known, for example, from WO 2008/017346. The preparation and storage of these compounds take place under inert conditions, for example with exclusion of oxygen, since the substances are oxidation-sensitive.

There is therefore an increasing demand for skin-tolerated and oxidation-stable compounds which are capable of functionalising protein-containing matrices and can be incorporated in a suitable manner into cosmetic or pharmacological preparations.

Surprisingly, it has now been established that the compounds of the formula I, as described below, are eminently suitable as protein-adhesive active compounds or in other words for the functionalisation of protein-containing matrices. The term protein-adhesive active compound is taken to mean an active compound which binds to a protein-containing matrix owing to at least one adhesion. Preferred matrices here are skin, hair and/or nails, where the general principle can also be applied to the functionalisation of synthetic polymer matrices containing amino groups or thiol groups, isolated proteins or gelatine. The products formed by adhesion and/or binding to such matrices can be used as, for example, cosmetic active compounds for the preparation of cosmetic compositions. The forces for adhesion which effect the cohesion have not been researched fully, and consequently there are various adhesion theories which encompass both mechanical adhesion owing to physical-mechanical forces and specific adhesion owing to chemical and physical forces. It is, for example, possible for the compounds of the formula I, as described below, to interact physically with the matrix molecules, as described above, due to their amphiphilicity or to form a covalent bond to an amino or thiol group of the matrix. The protein-adhesive active compounds can accordingly interact physically or form a covalent bond with the matrix. Due to the amphiphilicity, the compounds of the formula I, as described below, also have surface-active properties. The compounds of the formula I, as described below, are preferably skin- or hair-adhesive and/or skin- or hair-binding UV absorbers.

The compounds of the formula I, as described below, are likewise film-forming active compounds. Owing to the homogeneous distribution due to uniform surface adhesion and/or binding, an essential advantage, besides the long-lasting adhesion to the matrix, is that less active substance is required than in the case of the use of conventional UV absorbers.

The compounds of the formula I, as described below, are likewise suitable stabilisers for preparations, in particular cosmetic preparations or medicinal products, against UV radiation.

Compounds of the formula I according to the invention, as described below, are suitable as antiglycation agents and counter the formation of AGEs (advanced glycation end products).

Accordingly, the invention relates firstly to the use of compounds of the formula I,

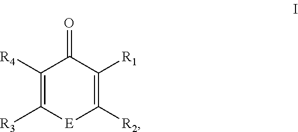

where
E denotes NR₅ or O,
R₁ denotes a UV chromophore, which is bonded via an O atom and renders the compound of the formula I capable of absorbing UV radiation in the range from 400 to 200 nm,
R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms
and/or salts, tautomers, stereoisomers and/or solvates thereof, including mixtures thereof in all ratios, as protein-adhesive active compounds, in particular as skin- or hair-adhesive and/or skin- or hair-binding UV absorbers.

This use is preferably a non-therapeutic use.

The invention furthermore also relates to the non-therapeutic use of compounds of the formula I,

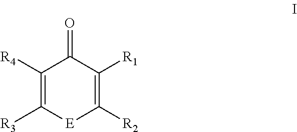

where
E denotes NR₅ or O,
R₁ denotes a UV chromophore, which is bonded via an O atom and renders the compound of the formula I capable of absorbing UV radiation in the range from 400 to 200 nm,
R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms and/or salts, tautomers, stereoisomers and/or solvates thereof, including mixtures thereof in all ratios, for protection of the skin, of the hair and/or of the nails against UV-induced damage.

For the purposes of the invention, the compounds of the formula I, are defined in such a way that they are also taken to mean pharmaceutically or cosmetically usable derivatives, salts, hydrates, solvates, precursors of the compounds, tautomers and optically active forms (such as, for example, stereoisomers, diastereomers, enantiomers, racemates). Solvates of the compounds are taken to mean adductions of inert solvent molecules onto the compounds which form owing to their mutual attractive force. Solvates are, for example, mono- or dihydrates or alcoholates. Pharmaceutically or cosmetically usable derivatives are taken to mean, for example, the salts of the compounds according to the invention. The compounds of the formula I can form cis/trans isomers or tautomers. The formula I encompasses all these forms.

A base of the formula I can be converted into the associated acid-addition salt using an acid, for example by reaction of equivalent amounts of the base and the acid in an inert solvent, such as ethanol, and subsequent evaporation. Suitable acids for this reaction are, in particular, those which give physiologically acceptable salts.

On the other hand, acids of the formula I can be converted into the corresponding metal salts, in particular alkali metal or alkaline-earth metal salts, or into the corresponding ammonium salts using bases (for example sodium hydroxide or potassium hydroxide or sodium carbonate or potassium carbonate).

In accordance with the invention, compounds of the formula I, as described above, are preferably used if

R₁ is a substituent of the formula II, III, IV, V, VI, VII, VIII, IX, X, XI, XII, XIII, XIV or XV,

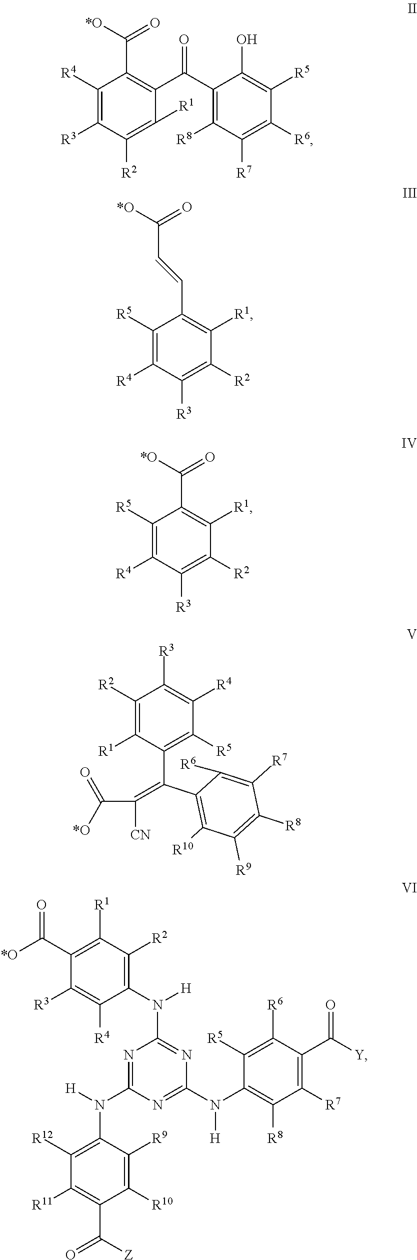

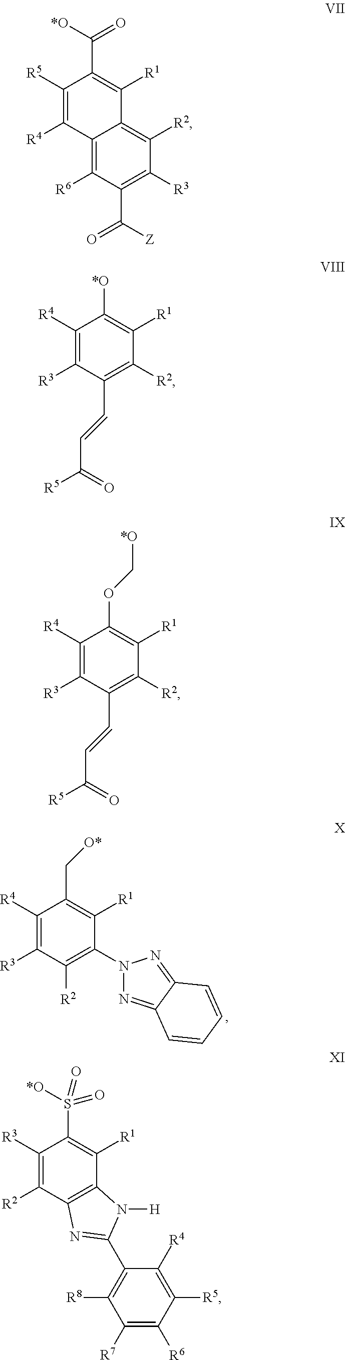

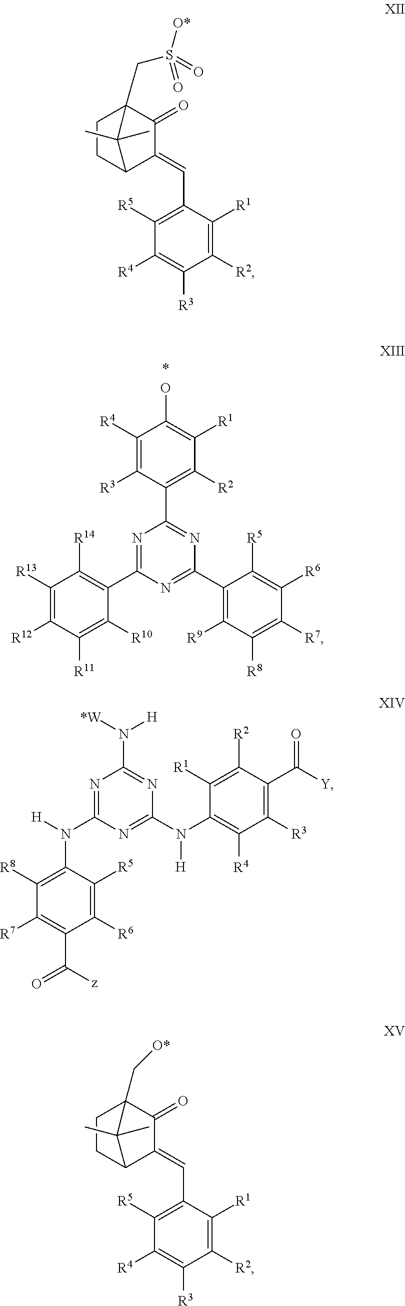

where
R¹ to R¹⁴ each, independently of one another, denote —H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or

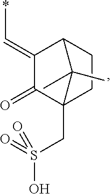 [SMILES: */C=C1/C(=O)C2(CS(=O)(=O)O)CCC1C2(C)C]

n is an integer from 1 to 25,
X is the counterion to the cations [NHA₂]⁺ and [NA₃]⁺ or the anion [SO₃]⁻ and Y and Z each, independently of one another, denote the structural unit I-1

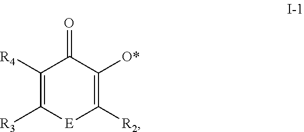

A, hydroxyl, —OA or —NH—C(CH₃)₃, where R₂, R₃, R₄ and E have a meaning given above for the compounds of the formula I, W denotes —(CH₂)ₘ—O or —(CH₂)ₒ—C(O)—O and
m and o each, independently of one another, denote an integer from 1 to 4, with the proviso that at least one substituent of the substituents R¹ to R¹⁴ in the formulae II, III, IV, VII, X, XII and XV denotes
OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or

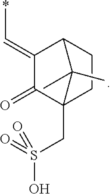 [SMILES: */C=C1/C(=O)C2(CS(=O)(=O)O)CCC1C2(C)C]

The invention furthermore relates to the compounds of the formula I,

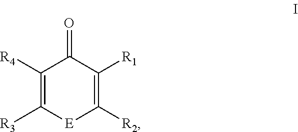

where
E denotes NR₅ or O,
R₁ is a substituent of the formula II, III, IV, V, VI, VII, VIII, IX, X, XI, XII, XIII, XIV or XV,

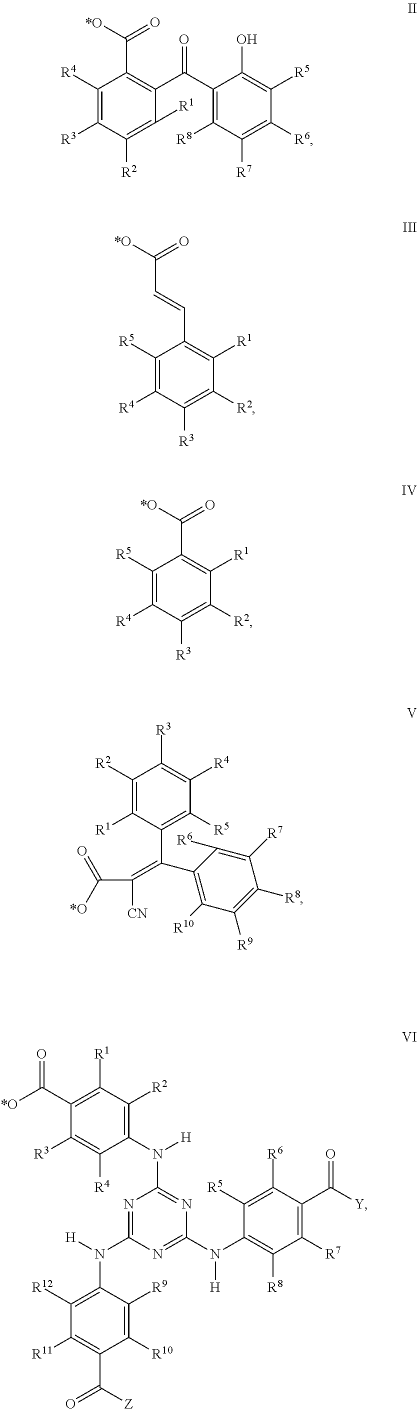

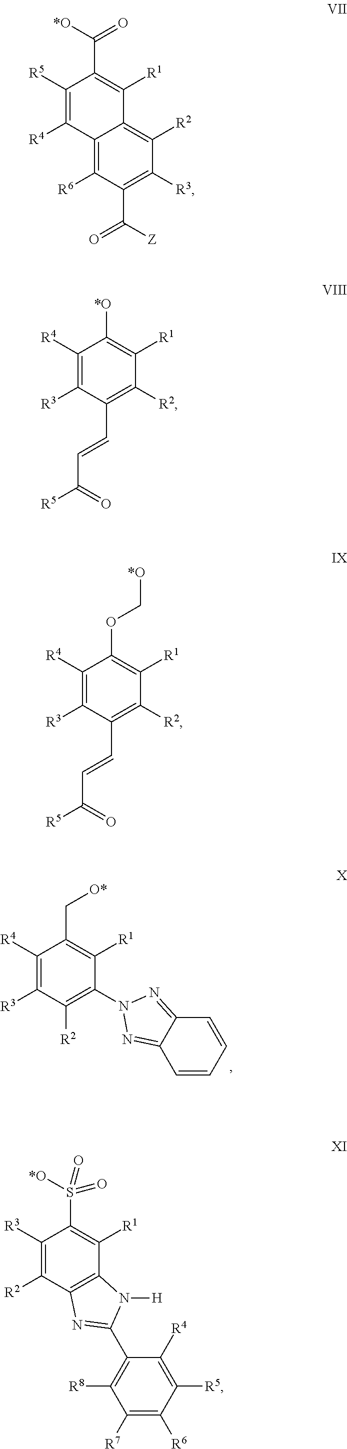

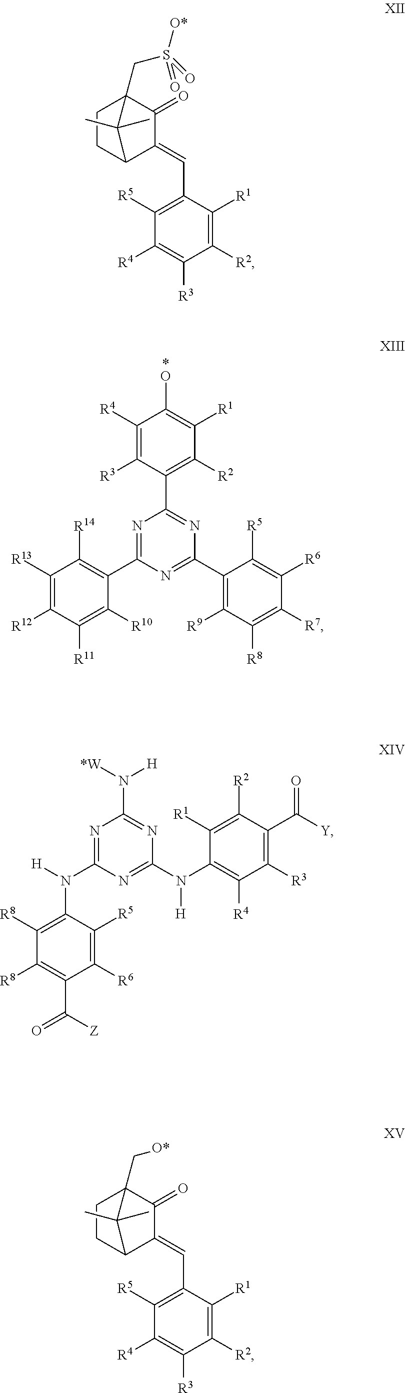

where
R¹ to R¹⁴ each, independently of one another, denote —H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or

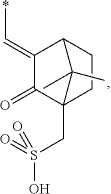 [SMILES: */C=C1/C(=O)C2(CS(=O)(=O)O)CCC1C2(C)C]

n is an integer from 1 to 25,
X is the counterion to the cations [NHA₂]⁺ and [NA₃]⁺ or the anion [SO₃]⁻ and
Y and Z each, independently of one another, denote the structural unit I-1

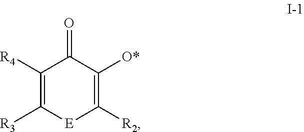

A, hydroxyl, —OA or —NH—C(CH₃)₃,
W denotes —(CH₂)ₘ—O or —(CH₂)ₒ—C(O)—O and
m and o each, independently of one another, denote an integer from 1 to 4, with the proviso that at least one substituent of the substituents R¹ to R¹⁴ in the formulae II, III, IV, VII, X, XII and XV denotes
OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or

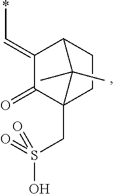 [SMILES: */C=C1/C(=O)C2(CS(=O)(=O)O)CCC1C2(C)C]

R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms,
where the compounds 2-methyl-4-oxo-4H-pyran-3-yl (E)-3-(4-hydroxy-phenyl)acrylate or 2-methyl-4-oxo-4H-pyran-3-yl 2-hydroxybenzoate are excluded.

The abbreviation A stands for alkyl having 1 to 20 C atoms, i.e. in other words for a linear or branched alkyl group having 1 to 20 C atoms, for example methyl, ethyl, isopropyl, propyl, butyl, sec-butyl or tert-butyl, furthermore pentyl, 1-, 2- or 3-methylbutyl, 1,1-, 1,2- or 2,2-dimethylpropyl, 1-ethylpropyl, n-hexyl, n-heptyl, n-octyl, furthermore nonyl, decyl, undecyl, dodecyl, tridecyl, tetradecyl, pentadecyl, hexadecyl, heptadecyl, octadecyl, nonadecyl and eicosyl. From the straight-chain or branched alkyl group having 1 to 20 C atoms, the straight-chain or branched alkyl group having 1 to 8 C atoms, i.e. methyl, ethyl, isopropyl, propyl, butyl, sec-butyl or tert-butyl, furthermore pentyl, 1-, 2- or 3-methylbutyl, 1,1-, 1,2- or 2,2-dimethylpropyl, 1-ethylpropyl, n-hexyl, n-heptyl, n-octyl, is preferably selected. From the straight-chain or branched alkyl group having 1 to 20 C atoms, the straight-chain or branched alkyl group having 1 to 4 C atoms, i.e. methyl, ethyl, isopropyl, propyl, butyl, sec-butyl or tert-butyl, is particularly preferably selected.

The abbreviation —OA stands for O-alkyl having 1 to 20 C atoms, analogously for alkoxy having 1 to 20 C atoms, where A is used as defined above. Correspondingly, the abbreviation —NHA stands for alkylamino and —NA₂ stands for dialkylamino.

The variable n stands for an integer from 1 to 25, preferably for an integer of 1, 2, 3, 4 or 5.

The variables m and o each stand, independently of one another, for 1, 2, 3 or 4. The variable m is preferably 2. The variable o is preferably 3.

X describes the counterion for the cations [NHA₂]⁺ and [NA₃]⁺, where A has one of the meanings indicated above, preferably Cl⁻, Br⁻, r or [SO₄]²⁻, or the counterion of the anion [COO]⁻ or [SO₃]⁻, preferably an ammonium ion or an alkali metal or alkaline-earth metal cation, such as Na⁺, K⁺, Mg²⁺ or Ca²⁺.

In an embodiment, compounds of the formula I in which the substituent R₄ denotes H are preferred or to be used preferably.

In an embodiment, compounds of the formula I in which the substituent R₃ denotes H, —(CH₂)ₚ—OH, —C(O)OA, COOH or COOX, where A, X and p have a meaning indicated above or preferably indicated, are preferred or to be used preferably. The substituent R₃ is particularly preferably H, hydroxy-methyl, —C(O)O-ethyl or —C(O)O-methyl. The substituent R₃ is very particularly preferably H.

In an embodiment, compounds of the formula I in which the substituent R₂ denotes A, where A has one of the meanings indicated above, are preferred or to be used preferably. The substituent R₂ is particularly preferably methyl or ethyl.

In an embodiment, compounds of the formula I in which the substituent R₅ denotes A and A has one of the meanings indicated as preferred are preferred or to be used preferably. R₅ is particularly preferably methyl or ethyl. R₅ is very particularly preferably methyl.

In a preferred embodiment, compounds of the formula I in which the variable E denotes O or NR₅ and the substituents R₅, R₄, R₃, R₂ and R₁ have one of the preferred meanings indicated above or indicated below or have meanings particularly preferably indicated are preferred or to be used preferably.

Accordingly, the compounds of the formulae I-a, I-b, I-c and I-d are particularly preferred:

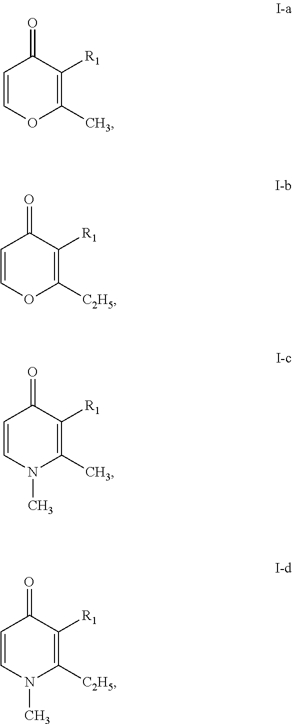

where R₁ has one of the meanings indicated below of the part-formulae listed or has one or more particularly preferred meanings of the part-formulae listed below.

In an embodiment, compounds of the formula I in which the variable E denotes O are preferred or to be used preferably.

In an embodiment, compounds of the formula I in which the variable E denotes N—R₅ and R₅ denotes A, where A has one of the meanings indicated above or preferably indicated, are preferred or to be used preferably. The variable E preferably denotes N-methyl.

In a preferred embodiment, compounds of the formula I in which the variable E denotes O and the substituents R₄, R₃, R₂ and R₁ have one of the preferred meanings indicated above or indicated below or have meanings which are particularly preferably indicated are preferred or to be used preferably. In this embodiment, the compounds of the formula I-a or I-b are particularly preferred. In this embodiment, the compounds of the formula I-a are very particularly preferred.

In a preferred embodiment, compounds of the formula I in which the variable E denotes NR₅ and the substituents R₅, R₄, R₃, R₂ and R₁ have one of the preferred meanings indicated above or indicated below or have meanings which are particularly preferably indicated are preferred or to be used preferably. In this embodiment, the compounds of the formula I-c or I-d are particularly preferred. In this embodiment, the compounds of the formula I-c are very particularly preferred.

Preferred embodiments of the substituent R₁ which is described by the formulae II, III, IV, V, VI, VII, VIII, IX, X, XI, XII, XIII, XIV and XV are listed below. Each preferred or particularly preferred embodiment of one of the said formulae II to XV can be combined with one or more of the said preferred or particularly preferred embodiments of the other formulae II to XV in order preferably to describe the substituent R₁ in the compounds of the formula I, I-a, I-b, I-c or I-d.

In an embodiment of the substituents of the formula II, R⁶ is preferably —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. Particularly preferably, the radicals R¹ to R⁵, R⁷ and R⁸ are H and

R⁶ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, very particularly preferably —OA, —NH₂, —NHA or —NA₂. From the list —OA, —NH₂, —NHA or —NA₂ indicated for R⁶, —OA or —NA₂ should preferably be selected. From the list —OA, —NH₂, —NHA or —NA₂ indicated for R⁶, —NA₂ should particularly preferably be selected. A has one of the meanings indicated above or meanings indicated as preferred. For R⁶, A preferably denotes a linear or branched alkyl group having 2 to 4 C atoms, particularly preferably ethyl, isopropyl, n-propyl, n-butyl, sec-butyl or tert-butyl.

In an embodiment of the substituents of the formula III, R³ is —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. Particularly preferably, the radicals R¹, R², R⁴ and R⁵ are H and

R³ is —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, very particularly preferably —OA, —NH₂, —NHA or —NA₂. From the list —OA, —NH₂, —NHA or —NA₂ indicated for R³, —OA or —NA₂ should preferably be selected. From the list —OA, —NH₂, —NHA or —NA₂ indicated for R³, —OA should particularly preferably be selected. A has one of the meanings indicated above or meanings indicated as preferred.

In an embodiment of the substituents of the formula IV, R³ or R⁵ are H, —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, where at least one substituent R³ or R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. Particularly preferably, the radicals R¹, R² and R⁴ are H and

R³ or R⁵ denote H, —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, where at least one substituent R³ or R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. R³ or R⁵ very particularly preferably denote H, —OH, —OA, —NH₂, —NHA or —NA₂, where at least one substituent R³ or R⁵ denotes —OH, —OA, —NH₂, —NHA or —NA₂. From the list —OH, —OA, —NH₂, —NHA or —NA₂ indicated for R³ or R⁵, —OA or —NA₂ should preferably be selected, in each case independently. From the list —OH, —OA, —NH₂, —NHA or —NA₂ indicated for R³ or R⁵, —NA₂ should particularly preferably be selected, independently in each case. A has one of the meanings indicated above or meanings indicated as preferred. In an embodiment, it is preferred if R⁵ denotes OH and R¹ to R⁴ are selected, in each case independently, from H, —OH, —OA, -A, —NH₂, —NHA, —NA₂, —NH—(CH₂—CH₂—O)ₙ—H, —N[(CH₂—CH₂—O)ₙ—H]₂ or 2H-benzotriazol-2-yl. In an embodiment, it is preferred if R⁵ denotes OH, R¹, R² and R⁴ denote H and R³ is selected from —OA, -A, —NH₂, —NHA, —NA₂, —NH—(CH₂—CH₂—O)ₙ—H, —N[(CH₂—CH₂—O)ₙ—H]₂ or 2H-benzotriazol-2-yl. In an embodiment, it is particularly preferred if R⁵ denotes OH, R¹, R² and R⁴ denote H and R³ is selected from —OA, -A, —NH₂, —NHA or —NA₂.

In an embodiment of the substituents of the formula IV, R² is 2H-benzotriazol-2-yl and R¹ or R³ are each, independently, preferably H, —OH, —OA, -A, —NH₂, —NHA, —NA₂, —NH—(CH₂—CH₂—O)ₙ—H or —N[(CH₂—CH₂—O)ₙ—H]₂. Particularly preferably, the radicals R⁴ and R⁵ are H and

R² denotes 2H-benzotriazol-2-yl and R¹ or R³ each, independently of one another, denote H, —OH, —OA, -A, —NH₂, —NHA, —NA₂, —NH—(CH₂—CH₂—O)ₙ—H or —N[(CH₂—CH₂—O)ₙ—H]₂, very particularly preferably —OH. Preferably, one substituent of R¹ or R³ is H and the other substituent is preferably —OH, —OA, -A, —NH₂, —NHA, —NA₂, —NH—(CH₂—CH₂—O)ₙ—H or —N[(CH₂—CH₂—O)ₙ—H]₂ or particularly preferably OH. A has one of the meanings indicated above or meanings indicated as preferred.

In an embodiment of the substituents of the formula V, the substituents R², R⁴, R⁷ and R⁹ are preferably H and the substituents R¹, R³, R⁵, R⁶, R⁸ and R¹⁰ are each, independently of one another, H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl. Preferably, R¹, R³, R⁵, R⁶, R⁸ and R¹⁰ in formula V are each, independently of one another, preferably H, —OA, —NH₂, —NHA or —NA₂

From the list —H, —OA, —NH₂, —NHA or —NA₂ indicated, preferably all substituents are H and/or R³ and/or R⁸ are —OA and R¹, R⁵, R⁶ and R¹⁰ are H. A has a meaning indicated above or a meaning indicated as preferred above.

In an embodiment of the substituents of the formula VI, the substituents R², R⁴, R⁵, R⁸, R⁹ and R¹² are preferably each, independently of one another, H or OH and the substituents R¹, R³, R⁶, R⁷, R¹⁰ and R¹¹ are each, independently of one another, H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and Y and Z preferably denote hydroxyl, —OA or —NH—C(CH₃)₃, where A has one of the meanings indicated above or meanings indicated as preferred. The substituents R¹, R³, R⁶, R⁷, R¹⁰ and R¹¹ in formula VI are each, independently of one another, preferably H, —OA, —NH₂, —NHA or —NA₂. The substituents R¹, R³, R⁶, R⁷, R¹⁰ and R¹¹ in formula VI are particularly preferably H. The variables Y and Z particularly preferably denote OA. The variables Y and Z very particularly preferably denote n-hexyloxy or 2-ethylhexyloxy.

In an embodiment of the substituents of the formula VII, the substituents R² and R⁴ are preferably H, at least one of the substituents R¹, R³, R⁵ and R⁶ is —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and Z has a meaning indicated above.

Z in formula VII preferably denotes —OH or —OA, where A has a meaning indicated above or has a meaning indicated as preferred.

In an embodiment of the substituents of the formula VII, the substituents R² and R⁴ are H, at least one of the substituents R¹, R³, R⁵ and R⁶ is preferably —OH or —OA and Z has a meaning indicated above.

In an embodiment of the substituents of the formula VII, the substituents R² and R⁴ are H, at least one of the substituents R¹, R³, R⁵ and R⁶ is —OH and Z has a meaning indicated above.

In an embodiment of the substituents of the formula VIII, R⁵ is preferably —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. Particularly preferably, the radicals R¹, R², R³ and R⁴ are H and

R⁵ denotes —OH, —OA, -A, —NH₂, —NHA, —NA₂, —NH—(CH₂—CH₂—O)ₙ—H, —N[(CH₂—CH₂—O)ₙ—H], SO₃H, SO₃X or 2H-benzotriazol-2-yl. R⁵ in formula VIII particularly preferably denotes —OA, where A has one of the meanings indicated above or meanings indicated as preferred. In compounds of the formula VIII, A particularly preferably denotes 2-ethylhexyl.

In an embodiment of the substituents of the formula IX, R⁵ is preferably —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl. Particularly preferably, the radicals R¹, R², R³ and R⁴ are H and

R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, very particularly preferably —OA. A has one of the meanings indicated above or meanings indicated as preferred. In compounds of the formula IX, A particularly preferably denotes 2-ethylhexyl.

In an embodiment of the substituents of the formula X, R¹ and/or R² are preferably —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl. Particularly preferably, the radicals R³ and R⁴ are H and

R¹ and/or R² denote —OH, —OA, -A, —NH₂, —NHA, —NA₂, —NH—(CH₂—CH₂—O)ₙ—H, —N[(CH₂—CH₂—O)ₙ—H]₂ or 2H-benzotriazol-2-yl, very particularly preferably —OH. A has one of the meanings indicated above or indicated as preferred.

In an embodiment of the substituents of the formula XI, R⁶ is preferably H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl. Particularly preferably, the substituents R¹, R², R³, R⁴, R⁵, R⁷ and R⁸ are preferably H and

R⁶ denotes H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl. A has one of the meanings indicated above or meanings indicated as preferred.

R⁶ in formula XI particularly preferably denotes H.

In an embodiment of the substituents of the formula XII, R³ is preferably —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or

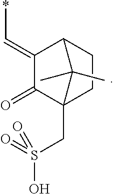 [SMILES: */C=C1/C(=O)C2(CS(=O)(=O)O)CCC1C2(C)C]

Particularly preferably, the substituents R¹, R², R⁴ and R⁵ are preferably H and
R³ denotes —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or

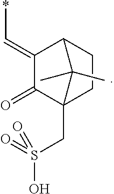 [SMILES: */C=C1/C(=O)C2(CS(=O)(=O)O)CCC1C2(C)C]

A has one of the meanings indicated above or meanings indicated as preferred.

R³ in formula XII particularly preferably denotes A.

In an alternative embodiment, compounds of the formula XII which contain the moiety

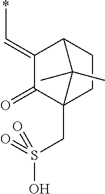 [SMILES: */C=C1/C(=O)C2(CS(=O)(=O)O)CCC1C2(C)C]

as substituent R³ are preferred.

In an embodiment of the substituents of the formula XIII, R⁵, R⁷, R¹² and R¹⁴ are each, independently of one another, preferably —OH, —OA, -A, —NH₂, —NHA, —NA₂, —NH—(CH₂—CH₂—O)ₙ—H, —N[(CH₂—CH₂—O)ₙ—H]₂ or 2H-benzotriazol-2-yl. Particularly preferably, the substituents R¹, R², R³, R⁴, R⁶, R⁸, R⁹, R¹⁰, R¹¹ and R¹³ are H and

R⁵, R⁷, R¹² and R¹⁴ each, independently of one another, denote —OH, —OA, -A, —NH₂, —NHA, —NA₂, —NH—(CH₂—CH₂—O)ₙ—H, —N[(CH₂—CH₂—O)ₙ—H]₂ or 2H-benzotriazol-2-yl. In an embodiment of the substituents of the formula XIII, the substituents R⁵ and R¹⁴ are OH or OA and R⁷ and R¹² are each, independently of one another, —OA and A has one of the meanings indicated above or indicated as preferred. In an embodiment of the substituents of the formula XIII, the substituents R⁵ and R¹⁴ are preferably OH and R⁷ and R¹² are each, independently of one another, OA and A has one of the meanings indicated above or indicated as preferred. A in formula XIII is preferably methyl or 2-ethylhexyl.

In an embodiment of the substituents of the formula XIV, W is preferably —CH₂—CH₂—O or —CH₂—CH₂—CH₂—C(O)O. Particularly preferably, the substituents R¹, R², R³, R⁴, R⁵, R⁶, R⁷ and R⁸ are H and

W is preferably —CH₂—CH₂—O or —CH₂—CH₂—CH₂—C(O)O. Y and Z in compounds of the formula XIV are preferably OA and A has one of the meanings indicated above. A in formula XIV is preferably n-butyl.

In an embodiment of the substituents of the formula XV, R³ is preferably —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or

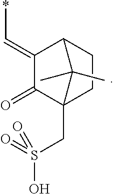 [SMILES: */C=C1/C(=O)C2(CS(=O)(=O)O)CCC1C2(C)C]

Particularly preferably, the substituents R¹, R², R⁴ and R⁵ are preferably H and
R³ denotes —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or

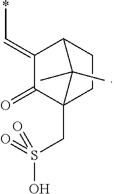 [SMILES: */C=C1/C(=O)C2(CS(=O)(=O)O)CCC1C2(C)C]

A has one of the meanings indicated above or meanings indicated as preferred.

R³ in formula XV particularly preferably denotes A, OA, NA₂ or

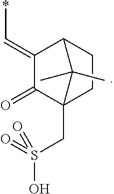 [SMILES: */C=C1/C(=O)C2(CS(=O)(=O)O)CCC1C2(C)C]

R³ in formula XV particularly preferably denotes A.

Further preferred combinations are disclosed in the claims. Preferred combinations are also combinations in which the preferred, particularly preferred or very particularly preferred meanings are used for each part-formula II to XV, in each case independently of one another.

Particularly preferred embodiments of the radical R₁ can be seen in the following moieties:

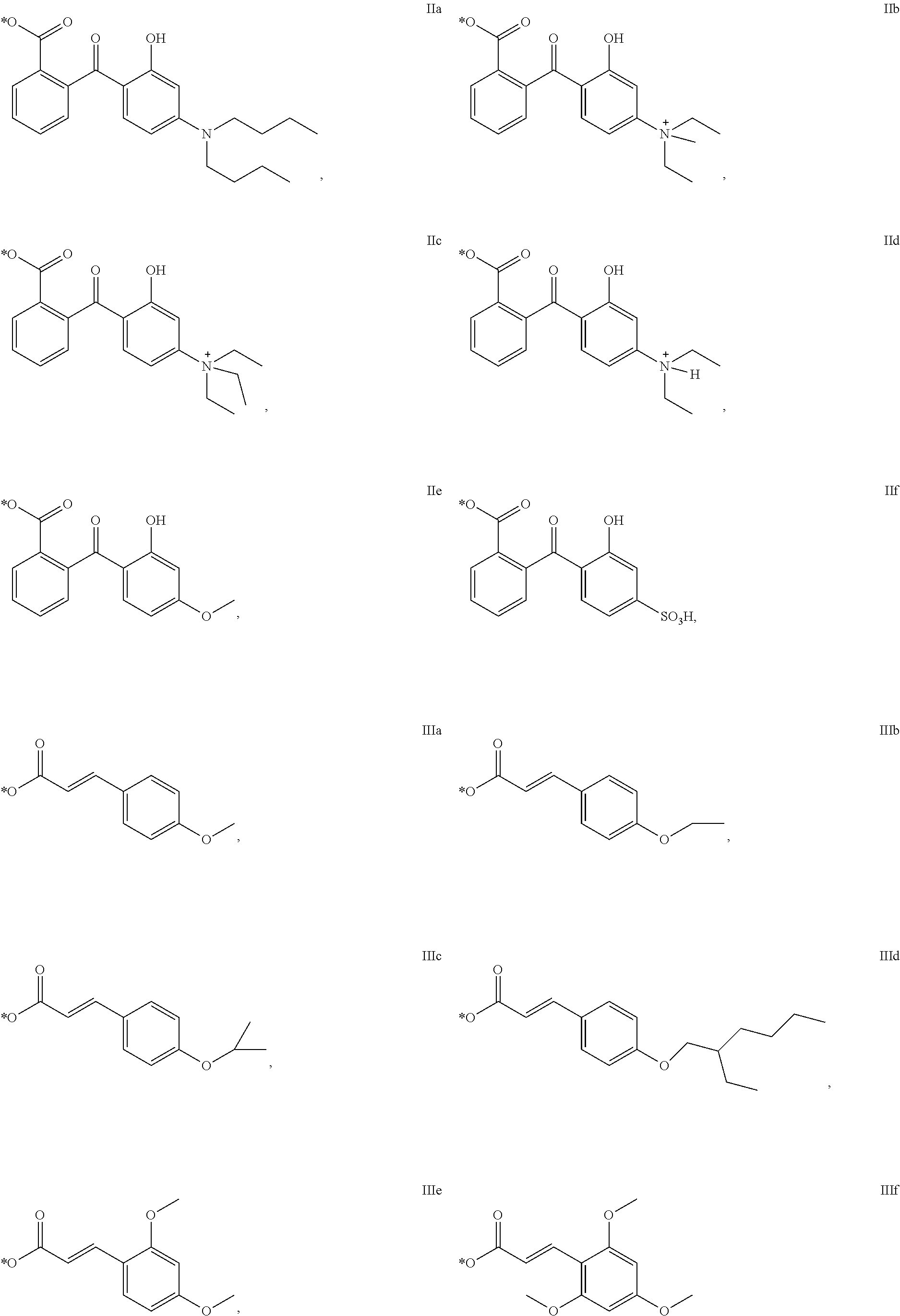

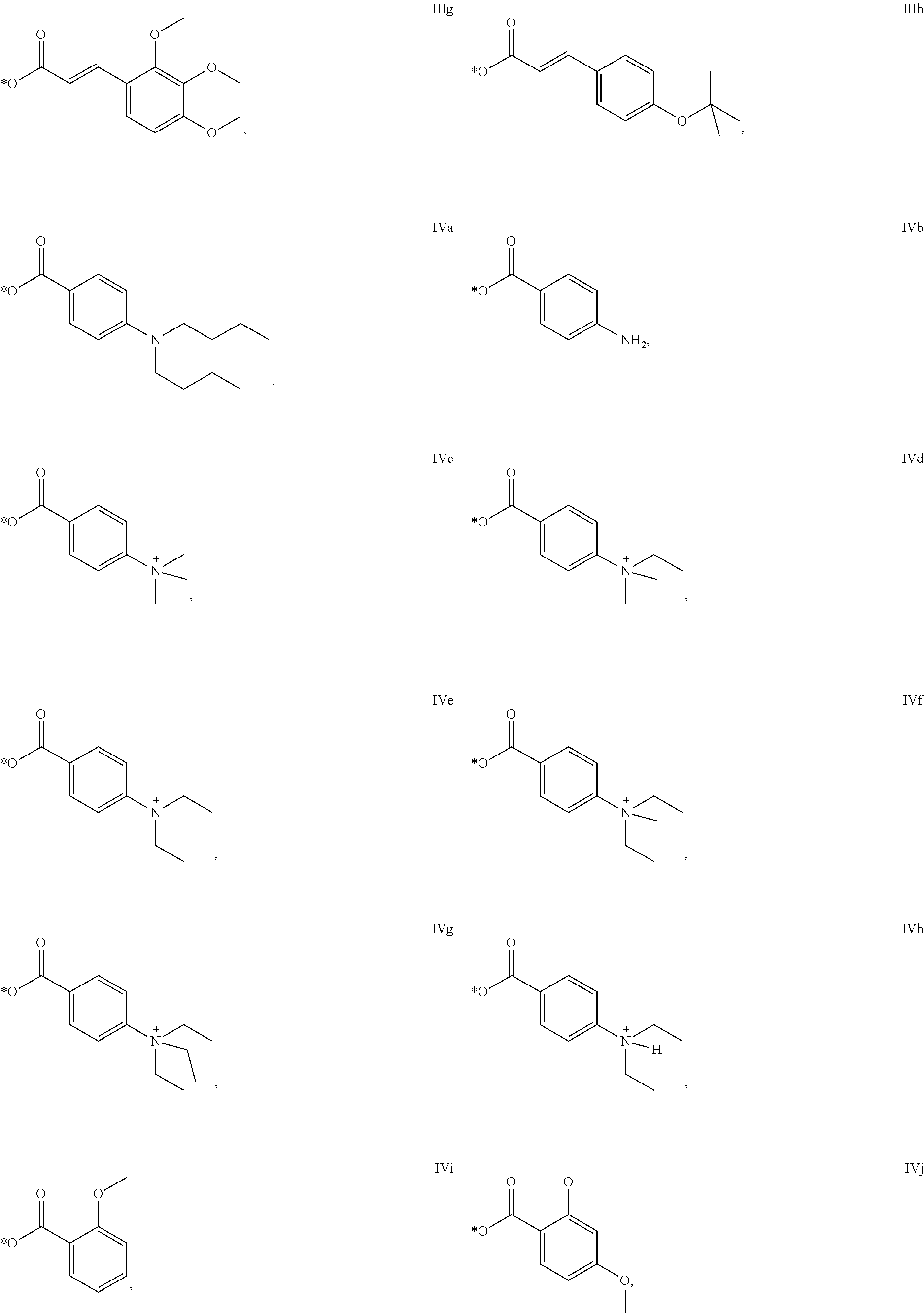

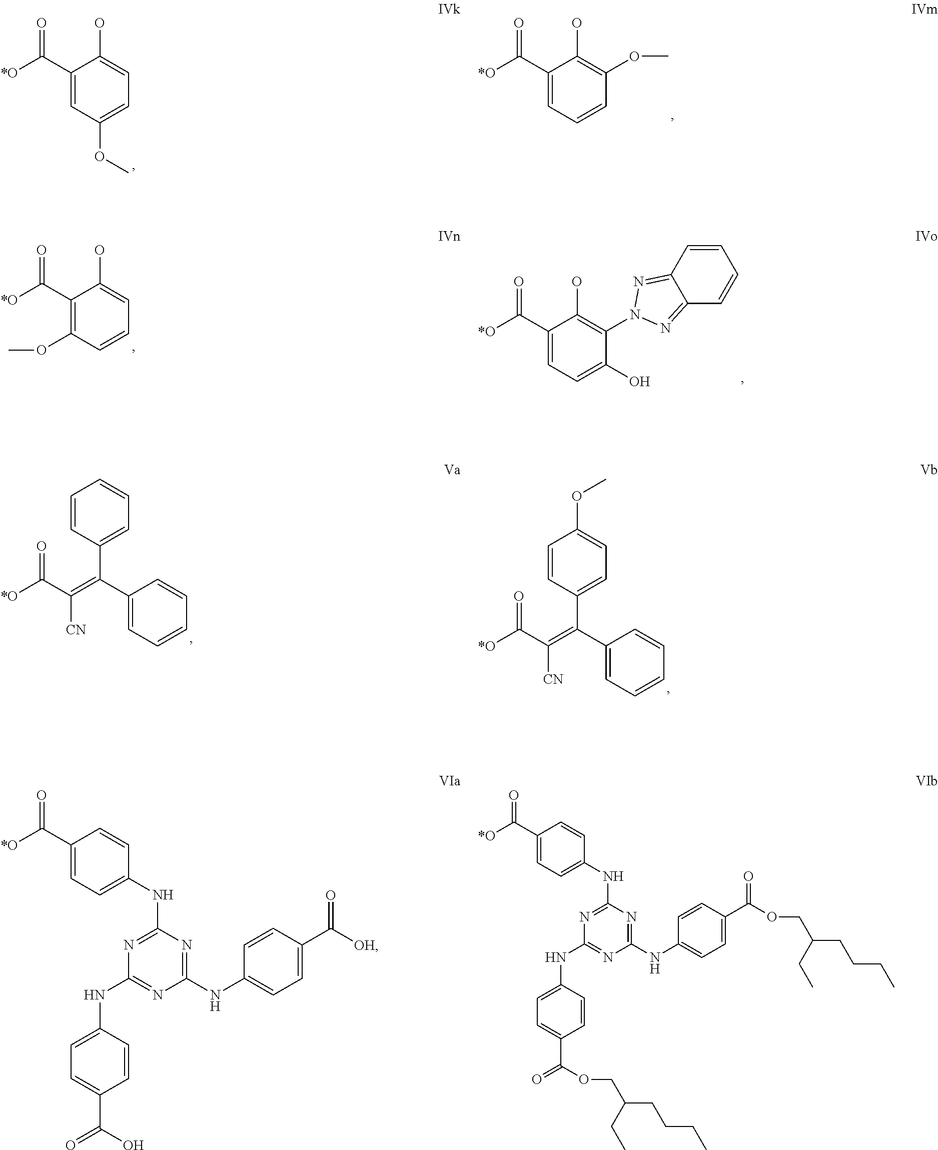

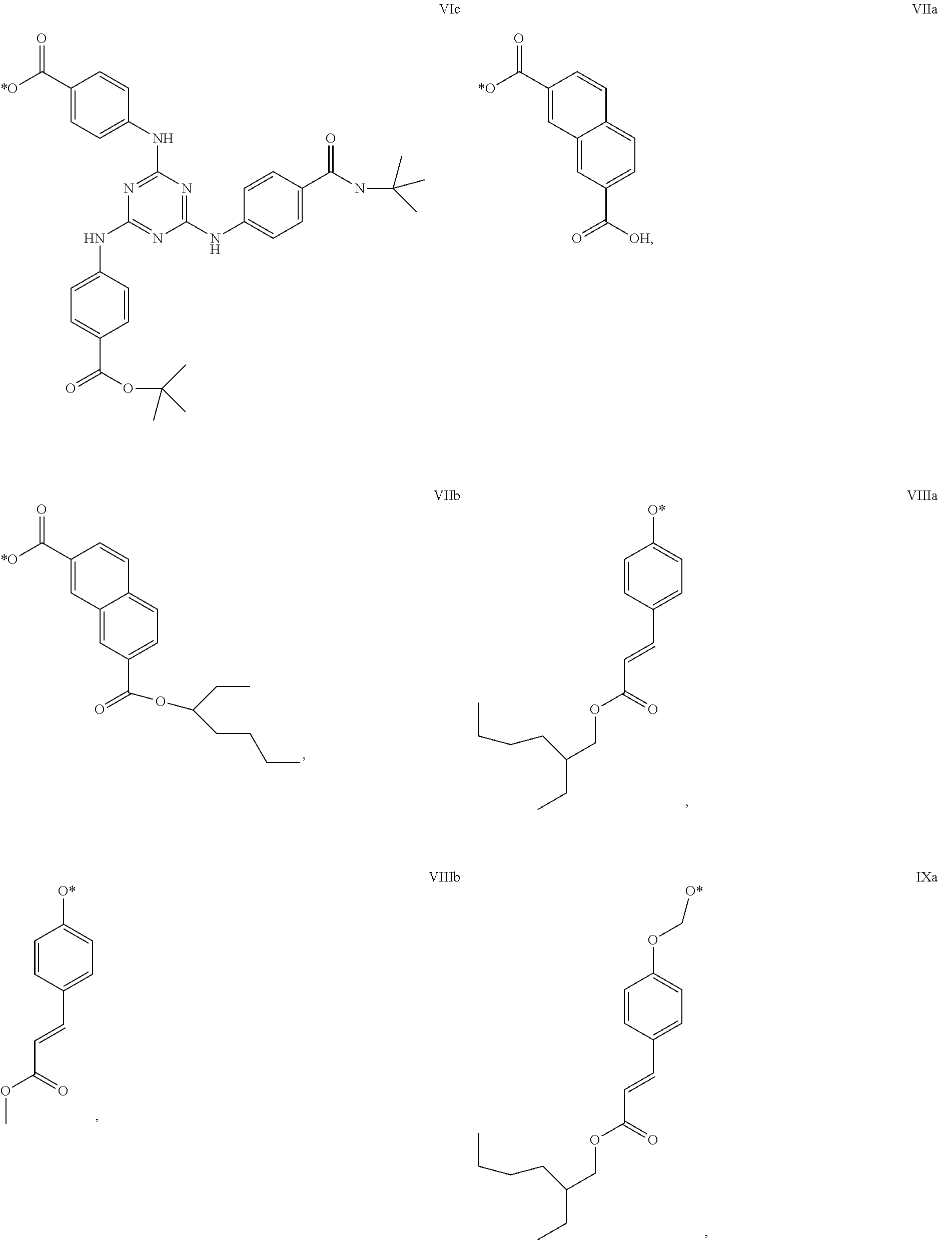

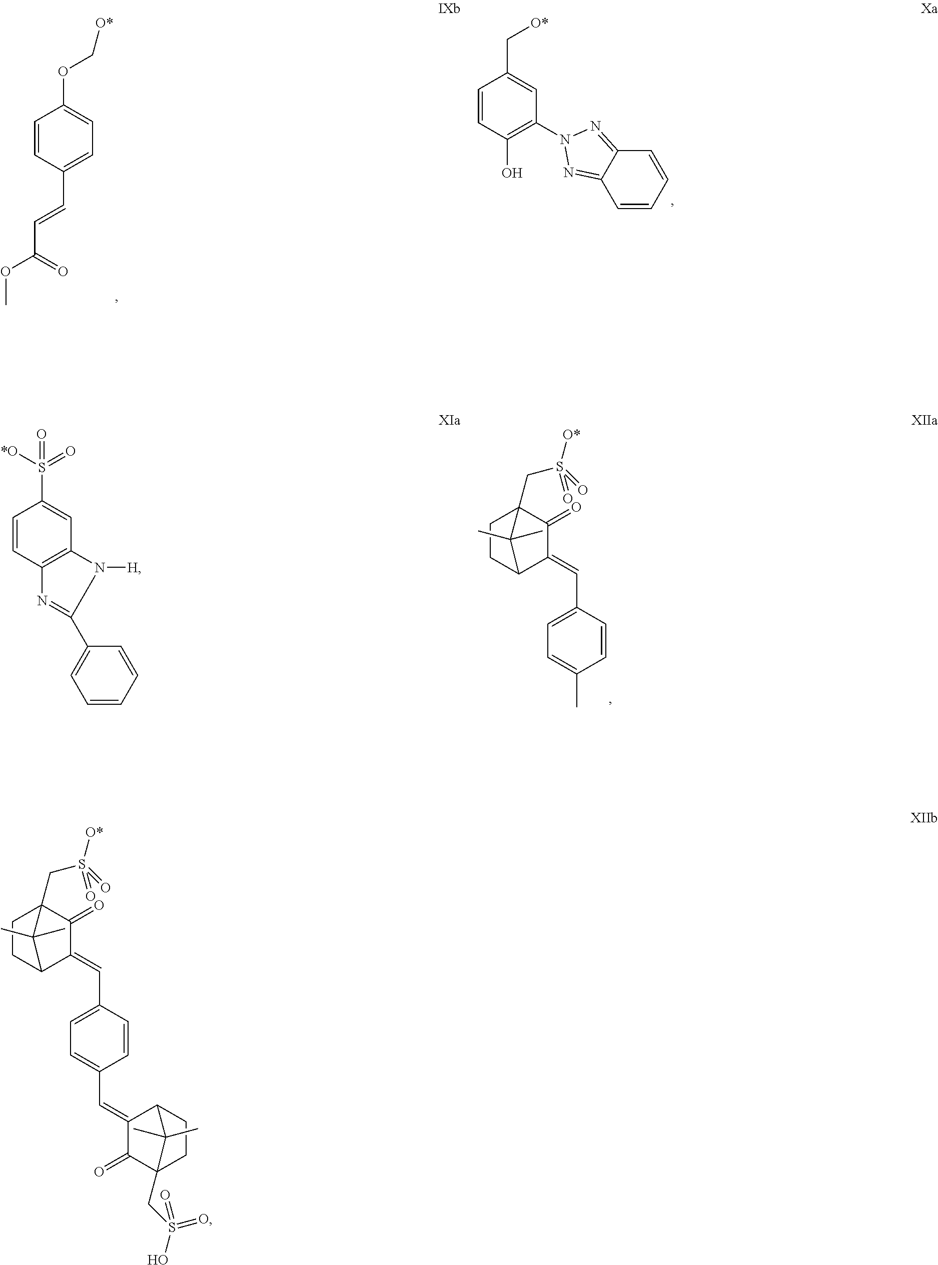

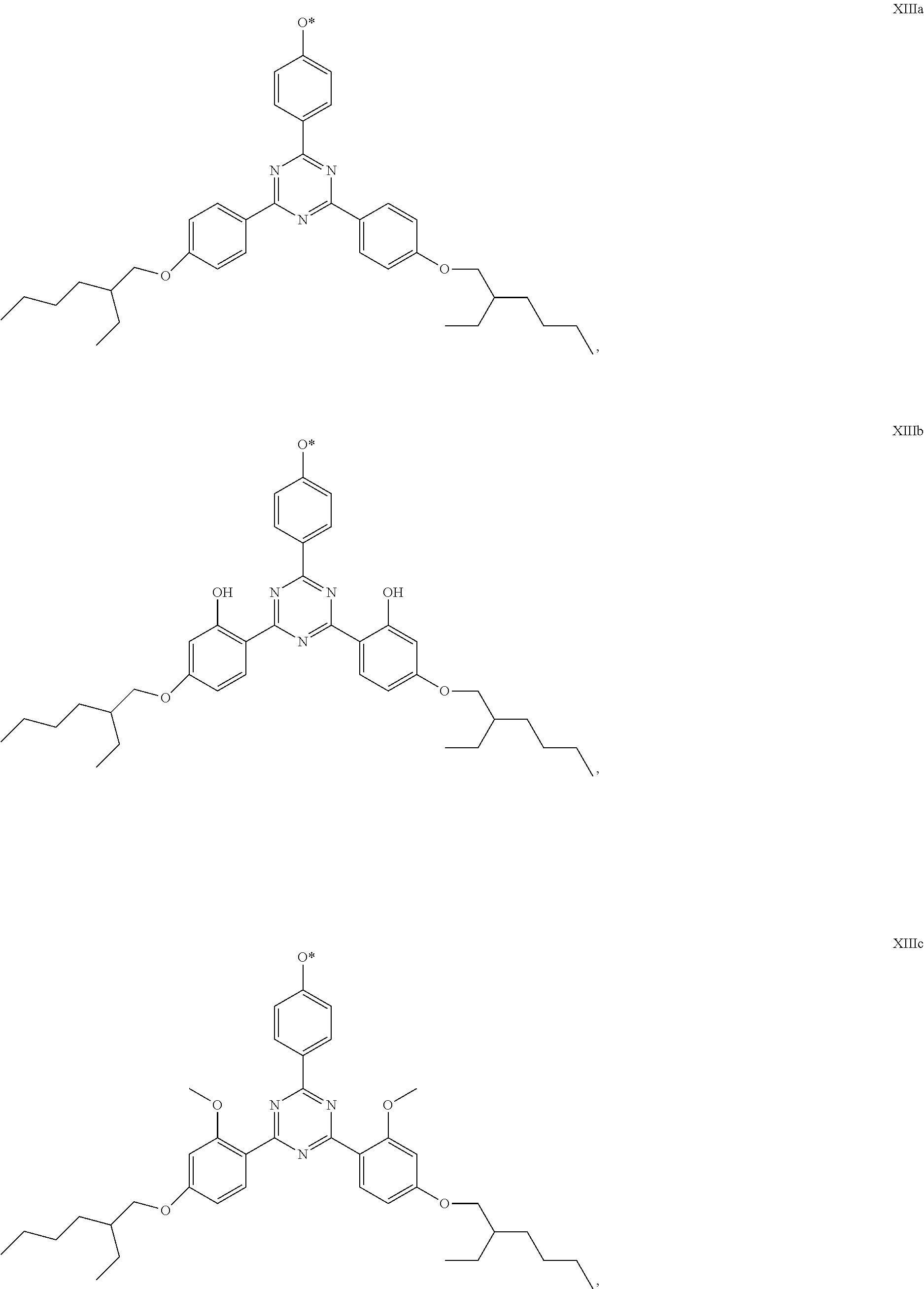

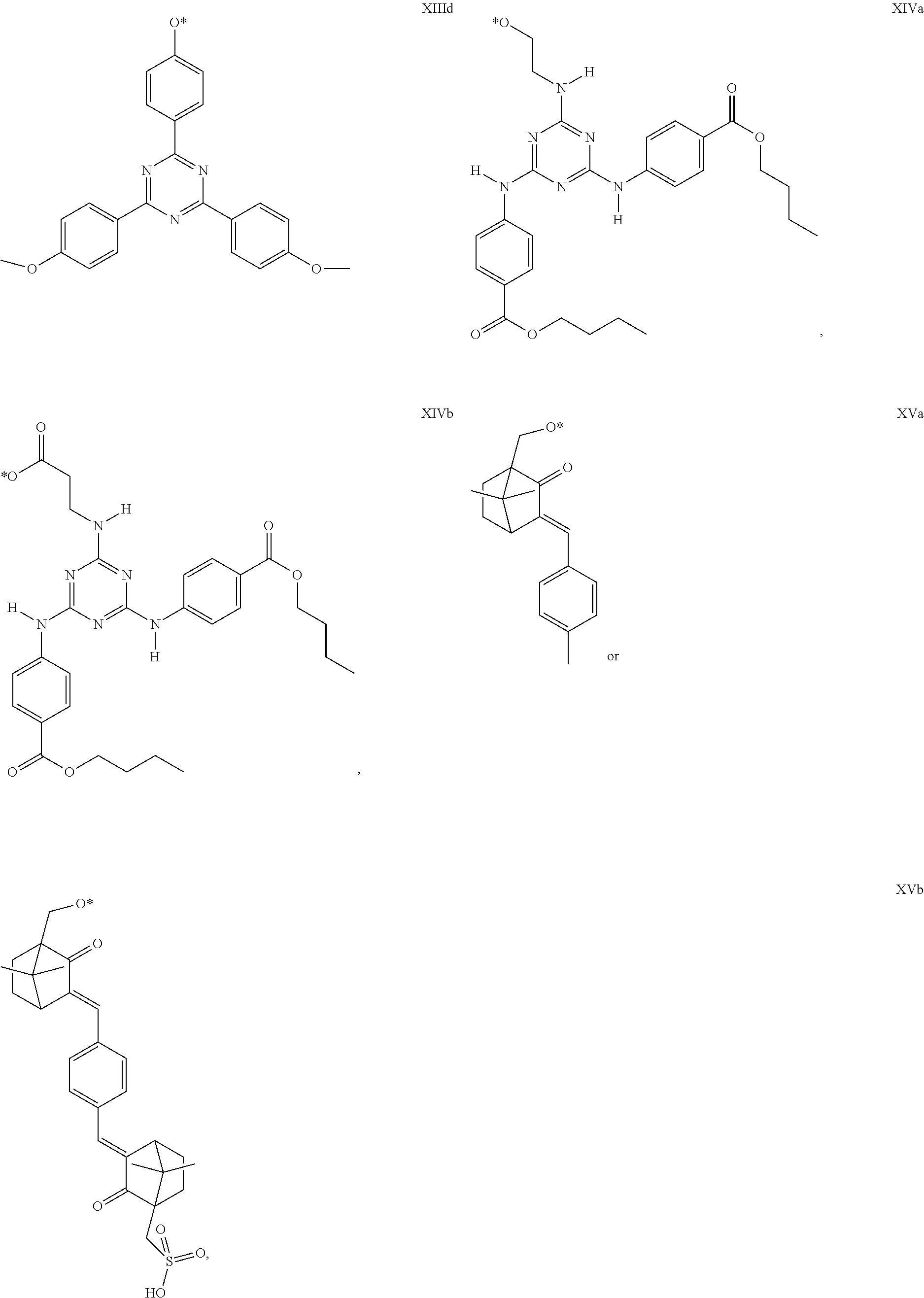

The radical R₁, as described above, is preferably bonded via a carbonyloxy function. R₁ therefore preferably conforms to the formula II, III, IV, V, VI or VII, where the radicals R¹ to R¹⁴ have one of the meanings given above or below or meanings preferably indicated or have the corresponding part-formulae.

R₁ very particularly preferably conforms to the formula II or III, where R¹ to R⁵ have a meaning indicated above or preferred meaning, in particular R₁ conforms to the part-formulae IIa to IIf and IIIa to IIIh, very particularly preferably to the part-formulae IIa and IIIa.

R₁ very particularly preferably conforms to the formula III, where R¹ to R⁵ have a meaning indicated above or preferred meaning, in particular to the part-formulae IIIa to IIIh, very particularly preferably to the part-formula IIIa.

The preparation of the compounds of the formula I, as described above, can be carried out here by methods known per se to the person skilled in the art from the literature. The reaction conditions for esterifications or etherifications are standard prior art and the selection of the suitable reaction conditions belongs to the standard expert knowledge of the person skilled in the art of synthesis.

The invention likewise relates to a process for the preparation of compounds of the formula I, as described above, characterised in that

a) a compound of the formula XVI

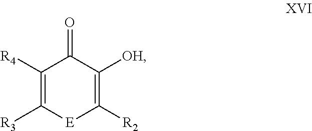

where E, R₂, R₃ and R₄ have a meaning indicated above,
is reacted with a compound of the formula XVII

R₁-M XVII,

in which R₁ has one of the meanings described above, where the part-formulae XI and XII are excluded, and
M denotes alkali metal or alkaline-earth metal cation or H or
b) a compound of the formula XVI

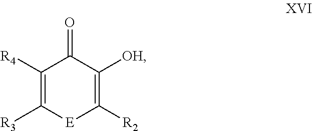

where E, R₂, R₃ and R₄ have a meaning indicated above,
is reacted with an acid halide or an active ester of the free acids derived from the formulae II, III, IV, V, VI, VII, XI, XII and XIV.

Some of the compounds of the formula XVI are commercially available, for example 3-hydroxy-2-methyl-4-pyranone, or can be prepared by known literature methods, for example based on R. Suzuki et al, Heterocyles, 1977, 6(9-10), 1575-80 or A. Fassihi et al, European Journal of Medicinal Chemistry, 2009, 44(5), 2145-2157).

Some of the compounds of the formula XVII or the free acids derived from the formulae II, III, IV, V, VI, VII, XI, XII and XIV are commercially available, for example p-methoxycinnamic acid, p-hydroxycinnamic acid, 2-(4-dimethylamino-2-hydroxybenzoyl)benzoic acid or 2-(4-methoxy-2-hydroxy-benzoyl)benzoic acid, or can be synthesised by methods which are described, for example, in the standard works, such as Houben-Weyl, Methoden der organischen Chemie [Methods of Organic Chemistry], Georg-Thieme-Verlag, Stuttgart, to be precise under reaction conditions which are known and suitable for the said reactions. Use can also be made here of variants known per se which are not mentioned here in greater detail.

The ether formation by reaction of compounds of the formula XVII in which R₁ conforms to the part-formulae VIII, IX, X, XIII, XIV or XV and in which M=H with a compound of the formula XVI, as described above, preferably takes place in the presence of triphenylphosphine and diisopropyl azodicarboxylate, in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of these solvents, at temperatures between about −10 and 40, preferably between 0 and 30°. The reaction time is between a few minutes and several days, depending on the conditions used.

If an acid halide or an active ester of the free acid derived from the formulae II, III, IV, V, VI, VII, XI, XII and XIV are employed for the synthesis of the compounds of the formula I in which R₁ corresponds to one of the moieties II, III, IV, V, VI, VII, XI, XII and XIV,

a classical nucleophilic substitution takes place. The reaction conditions of a nucleophilic substitution are adequately known to the person skilled in the art of synthesis.

Preferred acid halides are acid chlorides.

If a compound of the formula XVI, as described above, for example 3-hydroxy-2-methyl-4-pyranone, is reacted with a compound of formula XVII in which M=H and R₁ preferably conforms to the part-formula II, III, IV, V, VI, VII or XIV, the coupling reaction is preferably carried out in the presence of a dehydrating agent, for example a carbodiimide, such as dicyclohexylcarbodiimide (DCC), N-(3-dimethylaminopropyl)-N′-ethylcarbodiimide hydrochloride (EDC) or diisopropylcarbodiimide (DIC), furthermore, for example, propanephosphonic anhydride (cf. Angew. Chem. 1980, 92, 129), diphenylphosphoryl azide or 2-ethoxy-N-ethoxycarbonyl-1,2-dihydroquinoline, in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of these solvents, at temperatures between about −10 and 40, preferably between 0 and 30°. Depending on the conditions used, the reaction time is between a few minutes and several days.

Instead of compounds of the formula XVII, as defined above, it is also possible to employ derivatives of the formula XVII, preferably a pre-activated carboxylic acid or a carboxylic acid halide, a symmetrical or mixed anhydride or an active ester. Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart). Activated esters are advantageously formed in situ, for example by addition of HOBt (1-hydroxybenzotriazole) or N-hydroxy-succinimide.

The reaction is generally carried out in an inert solvent, in the case of the use of an acid halide in the presence of an acid-binding agent, preferably an organic base, such as triethylamine, dimethylaniline, pyridine, dimethyl-aminopyridine or quinoline.

The addition of an alkali or alkaline-earth metal hydroxide, carbonate or bicarbonate or of another salt of a weak acid of the alkali or alkaline-earth metals, preferably of potassium, sodium, calcium or caesium, may also be favourable.

The compounds of the formula I described are capable of adsorbing onto and/or binding to protein-containing textiles or textile fibres and thus developing their action, in each case depending on the radical R₁, for example UVB protection. Undesired material damage is thus reduced.

The compounds of the formula I according to the invention, as described above or as preferably described, are oxidation-stable and exhibit a reduced yellow colouration or no yellow colouration on storage.

The compounds of the formula I according to the invention, as described above or as preferably described, also have, for example, anti-ageing effects, i.e. they serve, for example, for skin regeneration and cause wrinkle reduction of (light)-aged skin, they furthermore increase, for example, the skin relief density, have a skin-taughtening action or strengthen, for example, the dermis-epidermis bond (papilla index). They protect the skin against UV-induced damage or they have, for example, a skin-bleaching action. They have, for example, an antibacterial action, i.e. they can reduce sweat odour or improve the skin appearance in the case of skin impurities and/or acne.

Compounds of the formula I according to the invention, as described above, are likewise suitable as anti-glycation agents and counter the formation of AGEs (advanced glycation end products).

Compounds according to the invention are suitable as contrast reduction agents, i.e. they may be able to lighten dark skin areas or darken light skin areas.

The compounds of the formula I according to the invention, as described above or as preferably described, are capable of bonding to hair and can thus suppress hair damage caused by UV light or by oxidation, in particular with respect to colour and morphology. For example, protection can thus be provided against bleaching of the hair.

The compounds of the formula I according to the invention, as described above or as preferably described, are capable of bonding not only to nitrogen-containing, but also to sulfur-containing hair functionalities, such as, for example, to thiolic groups.

The present invention furthermore relates to a preparation comprising at least one compound of the formula I, as described above or described as preferred or described as preferred individual compounds, where the compounds 2-methyl-4-oxo-4H-pyran-3-yl (E)-3-(4-hydroxyphenyl)acrylate or 2-methyl-4-oxo-4H-pyran-3-yl 2-hydroxybenzoate are excluded.

The invention furthermore relates to a preparation comprising 2-methyl-4-oxo-4H-pyran-3-yl (E)-3-(4-hydroxyphenyl)acrylate or 2-methyl-4-oxo-4H-pyran-3-yl 2-hydroxybenzoate, a cosmetic, dermatological or pharmacologically tolerated vehicle and at least one further active compound selected from the group UV filters, antioxidants, vitamins, anti-ageing active compounds, anticellulite active compounds, self-tanning substances, antipigmentation substances or skin-lightening substances.

Owing to the surface-adhesive, film-forming property of compounds according to the invention, further active compounds present in the preparation according to the invention can be protected particularly well against oxidative decomposition in preparations according to the invention, both before and during use thereof. Further active compounds of this type which are present in the composition according to the invention are, for example, dyes, in particular hair dyes and hair colorants, or other oxidation-sensitive substances, such as unsaturated fatty acids, squalene, vitamins, antioxidants, self-tanning substances, such as, for example, dihydroxyacetone, retinoids, such as, for example, retinol.

Furthermore, further active compounds present in the compositions according to the invention are optimally distributed on the action surface owing to the film-forming property of substances according to the invention and are thus enhanced in their action performance.

Compositions according to the invention can therefore be used, for example, as hair-treatment compositions for the
vitalisation of hair and/or
stimulation of energy metabolism in hair follicles and/or
activation of hair follicles and/or
promotion or enhancement of hair growth and/or
hair thickening and/or
treatment of hair loss and/or
influencing of keratin synthesis, in particular stimulation thereof, and/or
improvement of the tensile strength of keratinic fibres, in particular human hair, and/or
stabilisation of the moisture balance of keratinic fibres, in particular human hair, and/or
improvement of the combability of keratinic fibres, in particular human hair, and/or
retardation of the ageing process of keratinic fibres, in particular human hair, and/or
improvement of the restructurability of keratinic fibres, in particular human hair, and/or
reduction of the decrease in elasticity of keratinic fibres, in particular human hair, in the case of damage due to atmospheric effects.

The preparation here is usually a preparations which can be applied topically, for example cosmetic or dermatological formulations or medical devices. In this case, the preparations comprise a cosmetically or dermatologically suitable carrier and, depending on the desired property profile, optionally further suitable ingredients. In the case of pharmaceutical preparations, the preparations in this case comprise a pharmaceutically tolerated carrier and optionally further pharmaceutical active compounds. In the case of medical device, the preparations comprise a carrier which is suitable for the medical device.

Can be applied topically here means that the preparation is applied externally and locally, i.e. that the preparation must be suitable for, for example, application to the skin.

The topical preparations are preferably employed as cosmetic or dermatological preparation, particularly preferably as cosmetic preparation

The term composition is also synonymously taken to mean preparation or formulation.

The preparations may include or comprise, essentially consist of or consist of the said requisite or optional constituents. All compounds or components which can be used in the preparations are either known and commercially available or can be synthesised by known processes.

Preparations comprising 2-methyl-4-oxo-4H-pyran-3-yl (E)-3-(4-hydroxy-phenyl)acrylate or 2-methyl-4-oxo-4H-pyran-3-yl 2-hydroxybenzoate necessarily comprise a further active compound, as described above.

The compounds of the formula I or 2-methyl-4-oxo-4H-pyran-3-yl (E)-3-(4-hydroxyphenyl)acrylate or 2-methyl-4-oxo-4H-pyran-3-yl 2-hydroxybenzoate are typically employed in accordance with the invention in amounts of 0.01 to 20% by weight, preferably in amounts of 0.05% by weight to 10% by weight. The person skilled in the art is presented with absolutely no difficulties here in selecting the amounts correspondingly depending on the intended action of the preparation.

The functionalisation of the matrix or the evidence of binding can be checked using a few binding tests. These binding tests are described in the example part.

The preparations according to the invention preferably comprise at least one further active compound selected from the group UV filters, antioxidants, vitamins, anti-ageing active compounds, anticellulite active compounds, self-tanning substances, antipigmentation substances or skin-lightening substances.

The invention also relates to a process for the preparation of a preparation, as described above, in which at least one compound of the formula I as described above or as preferably described, is mixed with a carrier and optionally with further active compounds or assistants. Suitable carrier substances and active compounds or assistants are described in detail in the following part.

The mixing may result in dissolution, emulsification or dispersal of the compound of the formula I in the carrier.

Besides the compounds of the formula I and any other ingredients, the preparations may comprise further organic UV filters, so-called hydrophilic or lipophilic sun-protection filters, which are effective in the UVA region and/or UVB region and/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO 93/04665. Further examples of organic and also inorganic UV filters are indicated in the patent applications EP-A 0 487 404 and WO2009/077356. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed under the name “Escalol 507” by ISP, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, etocrylene, for example marketed by BASF under the name “Uvinul N35”. Furthermore, for example, methoxycrylene, marketed by Hallstar under the name Solastay S1.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethyl-amino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed under the name “Mexoryl SD” by Chimex, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidene-dicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: Ethylhexyltriazone, for example marketed by BASF under the name “Uvinul T150”, Diethylhexylbutamidotriazone, for example marketed by Sigma 3V under the name “Uvasorb HEB”. Further triazine derivatives are by way of example 2,4,6-tris(diisobutyl 4′-aminobenzal-malonate)-s-triazine or 2,4,6-tris(biphenyl)-1,3,5-triazine, or butyl 4-({4-{[4-(butoxycarbonyl)phenyl]amino}-6-[(3-{1,3,3,3-tetramethyl-1-[(trimethylsilyl)-oxy]disiloxanyl}propyl)amino]-1,3,5-triazin-2-yl}amino)benzoate, marketed under the name Mexoryl SBS. Structure of Mexoryl SBS:

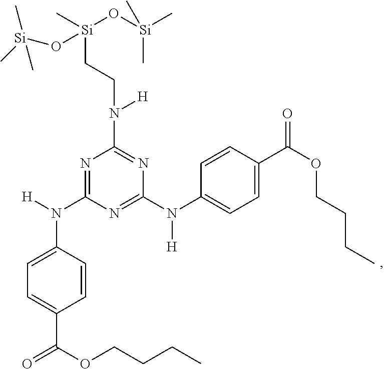 [SMILES: CCCCOC(=O)c1ccc(Nc2nc(NCC[Si](C)(O[Si](C)(C)C)O[Si](C)(C)C)nc(Nc3ccc(C(=O)OCCCC)cc3)n2)cc1]

and bis-ethylhexyloxyphenol methoxyphenyl triazine, for example marketed by BASF under the name Tinosorb S.

Anthraniline derivatives: Menthyl anthranilate, for example marketed by Symrise under the name “Neo Heliopan MA”.

Imidazole derivatives: ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

Piperazine derivatives, such as, for example, the compound

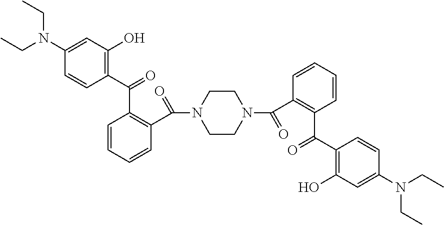 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

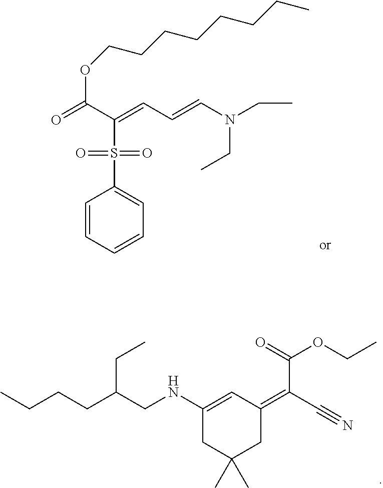

Preference is also given to a combination with UV filters based on poly-siloxane copolymers having a random distribution in accordance with the following formula, where, for example, a=1.2; b=58 and c=2.8:

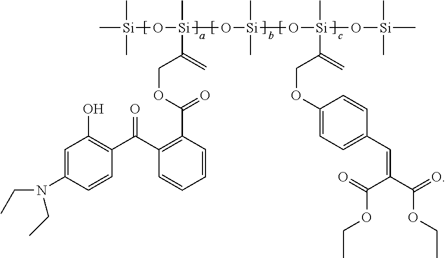

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethyl-amino-2-hydroxybenzoyl)benzoate, 4-Methylbenzylidenecamphor, Terephthalylidenedicamphorsulfonic acid, Disodium phenyldibenzimidazole-tetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-Bis[5-(1-dimethylpropyl)benzoxazol-2-yl(4-phenyl)-imino]-6-(2-ethylhexyl)imino-1,3,5-triazine and mixtures thereof.

These organic UV filters are generally incorporated into formulations in an amount of 0.01 percent by weight to 20 percent by weight, preferably 1% by weight—10% by weight.

The combination with organic UV filters, as described above, or inorganic UV filters, as described below, is particularly advantageous if the compounds of the formula I, as described above, are employed for the protection of skin and hair against photoageing by light.

Besides the compounds of the formula I and any other organic UV filters, as described above, the preparations may comprise further inorganic UV filters, so-called particulate UV filters.

These combinations with particulate UV filters are possible both as a powder and also as a dispersion or paste of the following types.

Preference is given here both to those from the group of the titanium dioxides, such as, for example, coated titanium dioxide (for example Eusolex® T-2000, Eusolex®T-AQUA, Eusolex®T-AVO, Eusolex®T-OLEO), zinc oxides (for example Sachtotec), iron oxides or also cerium oxides and/or zirconium oxides.

Furthermore, combinations with pigmentary titanium dioxide or zinc oxide are also possible, where the particle size of these pigments is greater than or equal to 200 nm, for example Hombitan® FG or Hombitan® FF-Pharma.

It may furthermore be preferred for the preparations to comprise inorganic UV filters which have been aftertreated by conventional methods, as described, for example, in Cosmetics & Toiletries, February 1990, Vol. 105, pp. 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerin.

Particulate UV filters which are preferably to be employed here are:
untreated titanium dioxides, such as, for example, the products Microtitanium Dioxide MT 500 B from Tayca; titanium dioxide P25 from Degussa,
Aftertreated micronised titanium dioxides with aluminium oxide and silicon dioxide aftertreatment, such as, for example, the product “Microtitanium Dioxide MT 100 SA” from Tayca; or the product “Tioveil Fin” from Uniqema,
Aftertreated micronised titanium dioxides with aluminium oxide and/or aluminium stearate/laurate aftertreatment, such as, for example, Microtitanium Dioxide MT 100 T from Tayca, Eusolex T-2000 from Merck,
Aftertreated micronised titanium dioxides with iron oxide and/or iron stearate aftertreatment, such as, for example, the product “Microtitanium Dioxide MT 100 F” from Tayca,
Aftertreated micronised titanium dioxides with silicon dioxide, aluminium oxide and silicone aftertreatment, such as, for example, the product “Microtitanium Dioxide MT 100 SAS” from Tayca,
Aftertreated micronised titanium dioxides with sodium hexametaphosphates, such as, for example, the product “Microtitanium Dioxide MT 150 W” from Tayca.

The treated micronised titanium dioxides to be employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerin; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2S13 from Cardre,
polydimethylhydrogenosiloxanes, such as, for example, the product Microtitanium Dioxide USP Grade Hydrophobic from Color Techniques.

The combination with the following products may furthermore also be advantageous:
Untreated zinc oxides, such as, for example, the product Z-Cote from BASF (Sunsmart), Nanox from Elementis
Aftertreated zinc oxides, such as, for example, the following products:
   “Zinc Oxide CS-5” from Toshibi (ZnO aftertreated with polymethyl-hydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes)
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilsesquioxane)
   Untreated cerium oxide micropigment, for example with the name “Colloidal Cerium Oxide” from Rhone Poulenc
   Untreated and/or aftertreated iron oxides with the name Nanogar from Arnaud.

For example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide, zinc oxide mixtures, such as, for example, the product UV-Titan M261 from Sachtleben, in combination with the UV protection agent according to the invention.

These inorganic UV filters are generally incorporated into the preparations in an amount of 0.1 percent by weight to 25 percent by weight, preferably 2% by weight—10% by weight.

By combination of one or more of the said compounds having a UV filter action, the protective action against harmful effects of the UV radiation can be optimised.

All the said UV filters can also be employed in encapsulated form. In particular, it is advantageous to employ organic UV filters in encapsulated form. It may therefore be preferred for one or more of the above-mentioned UV filters to be in encapsulated form. It is advantageous here for the capsules to be so small that they cannot be observed with the naked eye. In order to achieve the above-mentioned effects, it is furthermore necessary for the capsules to be sufficiently stable and the encapsulated active compound (UV filter) only to be released to the environment to a small extent, or not at all.

However, the protective action against oxidative stress or against the effect of free radicals can be improved further if the compositions or preparations according to the invention comprise one or more further antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homo-cysteine sulfoximine, buthionine sulfones, penta-, hexa- and heptathionine sulfoximine) in very low tolerated doses (for example pmol to μmol/kg), and also (metal) chelating agents (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

A further antioxidant or also a chelating agent is pentasodium ethylenediaminetetramethylenephosphonate.

Suitable antioxidants are also compounds of the general formulae A or B

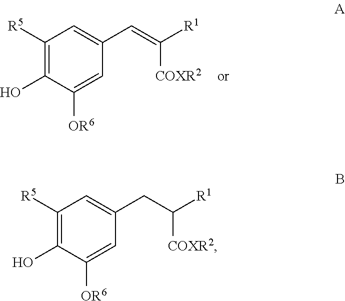

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes linear or branched alkyl having 1 to 8 C atoms or linear or branched alkoxy having 1 to 8 C atoms, and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxy-benzy)malonate (for example RonaCare® AP).

Mixtures of antioxidants are likewise suitable for use in the compositions or preparations according to the invention. Known and commercial mixtures are, for example, mixtures comprising, as active compounds, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with the compounds of the formula I in such compositions in ratios in the range from 1000:1 to 1:1000, preferably in amounts of 100:1 to 1:100.

The compositions or compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergo-calciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably retinol, nicotinamide, vitamin A palmitate, vitamin C and derivatives thereof, DL-α-tocopherol, tocopherol E acetate, nicotinic acid, pantothenic acid and biotin, very particularly preferably retinol or nicotinamide. Vitamins are usually employed here with compounds of the formula I in ratios in the range from 1000:1 to 1:1000, preferably in amounts of 100:1 to 1:100.

Suitable anti-ageing active compounds, in particular for skin-care preparations, are preferably so-called compatible solutes.

The compatible solutes employed are preferably substances selected from the group consisting of pyrimidinecarboxylic acids (such as ectoin and hydroxyectoin), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester, of these compounds, or combinations thereof.

Particularly preferred anti-ageing active compounds are pyrimidinecarboxylic acids, aryl oximes, bioflavonoids, bioflavonoid-containing extracts, chromones or retinoids.

Known bioflavonoids are, for example, troxerutin, tiliroside, α-glucosylrutin, rutin or isoquercetin, where the said choice is not intended to have a restrictive effect.

Bioflavonoid-containing extracts are, for example, ginkgo biloba or emblica.

Known anti-ageing substances are also chromones, as described, for example, in EP 1508327, or retinoids, for example retinol (vitamin A), retinoic acid, retinaldehyde or also synthetically modified compounds of vitamin A.

The chromones and retinoids described are simultaneously also effective anti-cellulite active compounds. A likewise known anti-cellulite active compound is caffeine.

Suitable anti-ageing active compounds are, for example, the products marketed by Merck 5,7-dihydroxy-2-methylchromone, marketed under the trade name RonaCare®Luremine or the products Ronacare®Isoquercetin, Ronacare®Tiliroside or Ronacare®Cyclopeptide 5.

Furthermore, the preparations according to the invention may comprise at least one self-tanning agent as further ingredient.

Advantageous self-tanning agents which can be employed are, inter alia: 1,3-dihydroxyacetone, glycerolaldehyde, hydroxymethylglyoxal, γ-dialdehyde, erythrulose, 6-aldo-D-fructose, ninhydrin, 5-hydroxy-1,4-naphtoquinone (juglone) or 2-hydroxy-1,4-naphtoquinone (lawsone). Very particular preference is given to 1,3-dihydroxyacetone, erythrulose or a combination thereof.

Preparations having self-tanner properties, in particular those which comprise dihydroxyacetone, tend towards malodours on application to the human skin, which are thought to be caused by degradation products of dihydroxyacetone itself or by products of side reactions and which are regarded as unpleasant by some users. It has been found that these malodours are prevented on use of formaldehyde scavengers and/or flavonoids. The preparation according to the invention comprising at least one self-tanner may therefore preferably also comprise formaldehyde scavengers and optionally flavonoids in order to improve the odour.

The preparations may also comprise one or more further skin-lightening active compounds or synonymously antipigmentation active compounds. Skin-lightening active compounds can in principle be all active compounds known to the person skilled in the art. Examples of compounds having skin-lightening activity are hydroquinone, kojic acid, arbutin, aloesin niacinamide, azelaic acid, elagic acid, tranexamic acid, potassium 4-methoxysalicylate, mulberry tree extract, magnesium ascorbyl phosphate, liquorice extract, emblica, ascorbic acid or rucinol, as well as substances as described under WO2007121845. Preparations of this type enable, for example, the skin contrast between light and dark areas to be reduced. The skin thus appears to be more homogeneously coloured. The appearance is younger than the strongly contrasted skin.

Suitable preparations are those for external application, for example can be sprayed onto the skin as cream or milk (O/W, W/O, O/W/O, W/O/W), as lotion or emulsion, in the form of oily-alcoholic, oily-aqueous or aqueous-alcoholic gels or solutions. They can be in the form of solid sticks or formulated as an aerosol. Administration forms such as capsules, dragees, powders, tablet solutions or solutions are suitable for internal use.

Assistants to be preferred originate from the group of preservatives, stabilisers, solubilisers, colorants, odour improvers.

The preservatives used are preferably approved preservatives which are listed in the Cosmetics Regulation, Annex 6, as positive list or also antimicrobial pigments, as described, for example, in WO 2004/0092283 or WO 2004/091567.

Suitable preservatives are therefore also alkyl esters of p-hydroxybenzoic acid, hydantoin derivatives, propionate salts or a multiplicity of ammonium compounds.

Very particularly preferred preservatives are methylparaben, propylparaben, imidazolidinylurea, sodium dehydroxyacetate or benzyl alcohol. Preservatives are employed in amounts between 0.5 and 2% by weight.

Emollients or softeners are often incorporated into cosmetic preparations. They are preferably employed in 0.5 to 50% by weight, preferably between 5 and 30% by weight, based on the composition as a whole. In general, softeners can be classified in classes, such as, for example, the category of the esters, fatty acids or fatty alcohols, polyols, hydrocarbons and oils containing at least one amide structural unit.

Representative oils containing at least one amide structural unit together with their synthesis are described, in particular, in EP 1044676 and EP 0928608. A compound which is particularly preferred indicated is isopropyl N-lauroylsarcosinate, which is commercially available from Ajinomoto under the product name Eldew SL-205.

Illustrative hydrocarbons as softeners are compounds which generally have 12 to 30 C atoms. Specific examples are arylalkyl benzoates, alkyl benzoates, mineral oils, Vaseline, squalenes or isoparaffins.

Further emollients or hydrophobicising agents are preferably C₁₂ to C₁₅ alkyl benzoates, dioctyl adipate, octyl stearate, octyldodecanol, hexyl laurate, octyldodecyl neopentanoate, cyclomethicone, dicaprylic ether, dimethicone, phenyltrimethicone, isopropyl myristate, caprylic/capric glyceride, propylene glycol dicaprylate/dicaprate or decyl oleate.

A further category of functional ingredients of cosmetic preparations in the sense of the invention are thickeners. Thickeners are generally employed in amounts between 0.1 to 20% by weight, preferably between 0.5 to 10% by weight, based on the total amount. Examples of these compounds are crosslinked polyacrylate materials, commercially available from B. F. Goodrich Company under the trade name Carbopol. It is also possible to use thickeners such as xanthan gum, carrageenan gum, gelatine gum, karaya gum, pectin gum or carob seed flour.

Under certain circumstances, it is possible for a compound to be both a thickener and also a softener. Examples thereof are silicone gums (kinematic viscosity>10 centistokes), esters, such as, for example, glycerol stearate, or cellulose derivatives, for example hydroxypropylcellulose.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexane-carbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, for example fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes, and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The preparations may preferably comprise assistants, such as, for example, cosmetic oils (for example Caprylic/Capric Triglycerides, C12-15 Alkyl Benzoate, isopropyl myristate, Arylalkyl Benzoate, such as, for example, phenethyl benzoate (X-Tend 226), isopropyl N-dodecanoyl-N-methylglycinate (Eldew SL 205), phytosteryl otyldodecyl lauroyl glutamate (Eldew PS 203) or oil components of the Cosmacol brand, such as Dimyristyl Tartrate, Tri C14-C15 Alkyl Citrate, C12-C13 Alkyl Lactate, Tridecyl Salicylate, C12-C13 Alkyl Octanoate, C12-C13 Alkyl Malate, C12-C13 Alkyl Citrate, C12-C13 Alkyl Tartrate), or polar-protic assistants (for example propylene glycol, glycerine, isopropanol, ethanol) or so-called solubilisers (for example butyl-phthalimides, isopropylphthalimides, dimethylisosorbides).

The oil phase may furthermore advantageously be selected from the group of branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty substances, as well as water and an emulsifier, as usually used for a formulation of this type.

In a preferred embodiment, the preparations to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The cosmetic and dermatological preparations may exist in various forms. Thus, they may be, for example, a solution, a water-free preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A-43 08 282, have proven favourable. Preference is given to emulsions. O/W emulsions are particularly preferred. Emulsions, W/0 emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alchols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth1-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention: fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in preparations of this type, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty substance, a lower monoalcohol or a lower polyol or mixtures thereof. Particularly preferred monoalcohols or polyols include ethanol, i-propanol, propylene glycol, glycerol and sorbitol.

A preferred embodiment is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, use is generally made of the customary propellants, such as alkanes.

The invention furthermore also relates to the use of the structural unit of the formula I-1

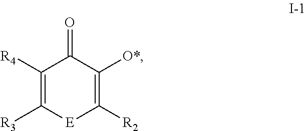

in which R₂, R₃, R₄ and E have a meaning indicated in the case of the compounds of the formula I or a meaning preferably indicated, as linker for the functionalisation of protein-containing matrices, where the symbol * denotes the linking site in the form of a covalent single bond to a molecule which is intended to be adsorbed onto or covalently bonded to the matrix.

The structural unit of the formula I-1 is essential for the adhesion and/or binding of the compounds of the formula I, as described above. The nature of the covalent single bond by means of which the molecule to be adsorbed or bonded is bonded to the linker is independent of the adhesion and/or binding to the protein-containing matrix. This linker of the formula I-1 provides molecules of this type, for example active compounds, with a surface-adhesive film-forming property and thus enables them to be adsorbed very uniformly onto suitable surfaces. Thus, for example, active compounds having a variety of structures can be bonded to the protein-containing matrix, in particular to the skin, hair and/or nails. This results, for example, in immobilisation of the desired active compounds, for example of UV absorbers, UV filters, dyes, such as, for example, fluorescent dyes for marking tissue, or enables controlled release of active compound, for example for pharmacological, antimicrobial, fungicidal, herbicidal, insecticidal or cosmetic active compounds or for X-ray contrast compositions.

This linker of the formula I-1 likewise provides molecules of this type, for example the class of organic UV absorbers or UV filters, with an amphiphilic property and they thus have a surface activity which, although not characterising them as a surfactant, does, however, help the active compounds to attain a property profile which differs from the property profile of classical, in particular oil-soluble, organic UV absorbers or UV filters.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below are incorporated into this application by way of reference. The percent by weight ratios of the individual ingredients in the preparations of the examples expressly belong to the disclosure content of the description and can therefore be utilised as features.

Further important features and advantages of the invention arise from the sub-claims and from the examples.

It goes without saying that the features mentioned above and the features still to be explained below can be used not only in the combination indicated in each case, but also in other combinations or individually without leaving the scope of the present invention.

Preferred embodiments of the invention are described in the examples and are explained in greater detail in the following description without restricting the scope of the present invention.

### EXAMPLES

### Example 1

### Synthesis of 2-methyl-4-oxo-4H-pyran-3-yl 2-(4-dibutylamino-2-hydroxybenzoyl)benzoate

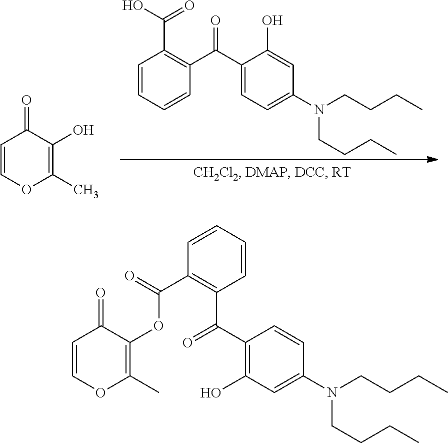

15.0 g of 2-(4-dibutylamino-2-hydroxybenzoyl)benzoic acid, 6.1 g of 3-hydroxy-2-methyl-4-pyranone and 0.35 g of 2-(dimethylamino)pyridine are initially introduced in 120 ml of dichloromethane under argon. A solution of 10.0 g of N,N′-dicyclohexylcarbodiimide (DCC) in 30 ml of dichloromethane is added dropwise to this solution, and the mixture is stirred at room temperature for 2 days. In order to eliminate excess DCCs, 4.4 g of oxalic acid dihydrate are then added, and the mixture is left to stir at room temperature for a further 2.5 h. The reaction solution is cooled in an ice-water bath for 30 min and subsequently filtered off. 100 ml of water are then added to the mother liquor, and the mixture is extracted. The organic phase is subsequently rinsed again with water, then with saturated NaCl solution, dried using Na₂SO₄, filtered and evaporated in a rotary evaporator.

Re-uptake in ethyl acetate and purification of the solution on silica gel gives 7.9 g of 2-methyl-4-oxo-4H-pyran-3-yl 2-(4-dibutylamino-2-hydroxybenzoyl)-benzoate.

¹³C-NMR (300 MHz, D₆-DMSO):): δ [ppm]=13.7; 14.3; 19.5; 28.8; 49.9; 96.5; 104.4; 108.9; 115.8; 126.6; 128.2; 129.8; 130.6; 133.3; 134.2; 137.7; 140.5; 154.2; 156.0; 159.2; 162.4; 164.7; 170.7; 186.5.

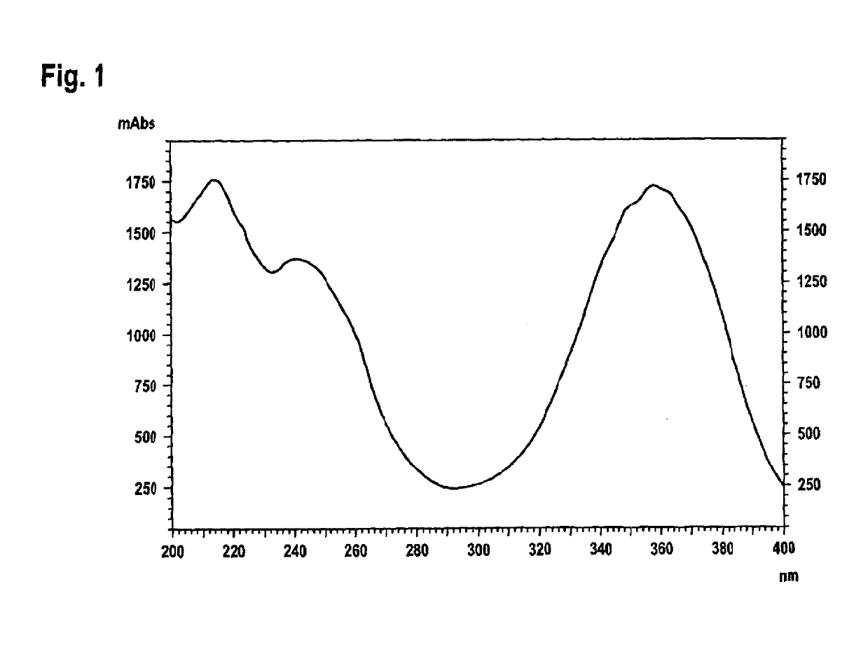

FIG. 1 shows the UV spectrum (200 to 400 nm) of the compound 2-methyl-4-oxo-4H-pyran-3-yl 2-(4-dibutylamino-2-hydroxybenzoyl)benzoate.

### Example 2

### Synthesis of 2-methyl-4-oxo-4H-pyran-3-yl 2-(E)-3-(4-methoxyphenyl)acrylate

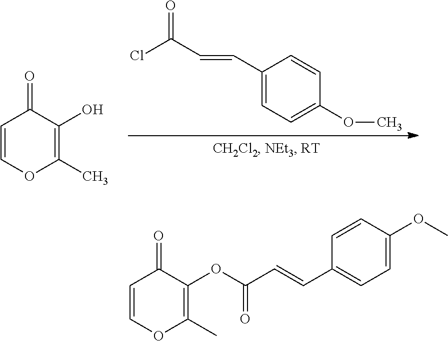

2.4 g of 3-hydroxy-2-methyl-4-pyranone are dissolved in 50 ml of dichloromethane at room temperature under argon. 4.5 g of trans-4-methoxycinnamoyl chloride are subsequently introduced, and 3.17 ml of triethylamine are slowly added dropwise. The mixture is left to stir at room temperature for 4 h. For filtration, the reaction solution is cooled to 0° C. The mother liquor is extracted with 25 ml of 1N HCl at room temperature. The organic phase is subsequently rinsed again with water, dried using Na₂SO₄, filtered, and the solvent is distilled off. For further purification, the residue is suspended in 15 ml of i-propanol, warmed briefly and stored in the refrigerator at 8° C. overnight. Suction filtration, rinsing and drying gives 4.8 g of 2-methyl-4-oxo-4H-pyran-3-yl 2-(E)-3-(4-methoxyphenyl)acrylate.

1H-NMR (300 MHz, D₆-DMSO): δ [ppm]=2.29 (s, 3H); 3.85 (s, 3H); 6.46 (d, 1H); 6.71 (d, 1H); 7.03 (d, 2H); 7.76 (d, 2H); 7.86 (d, 1H); 8.12 (d, 1H).

¹³C-NMR (300 MHz, D₆-DMSO):): δ [ppm]=14.5; 55.4; 113.2; 114.5; 114.5; 115.9; 126.3; 130.6; 131.0; 137.8; 147.0; 155.9; 159.2; 161.6; 163.3; 171.2.

### Example 3

### Synthesis of 2-methyl-4-oxo-4H-pyran-3-yl 3-(benzotriazol-2-yl-4-hydroxy)-benzoate

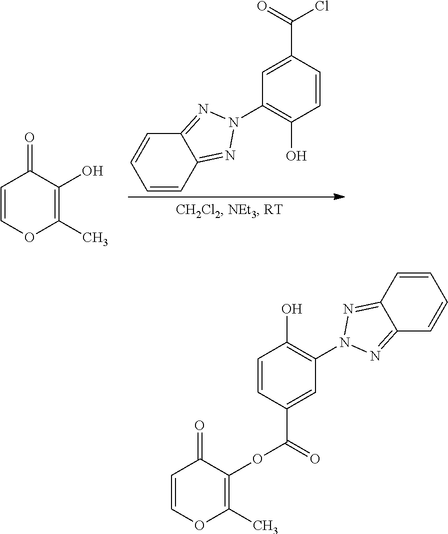

2.5 g of 3-hydroxy-2-methyl-4-pyranone are reacted with 7.5 g of benzotriazol-2-yl-4-hydroxybenzoyl chloride analogously to Example 2, giving 2-methyl-4-oxo-4H-pyran-3-yl 3-(benzotriazol-2-yl-4-hydroxy)benzoate.

### Example 4

### Synthesis of 2-ethylhexyl (E)-3-[4-(2-methyl-4-oxo-4H-pyran-3-yloxy)-phenyl]acryate

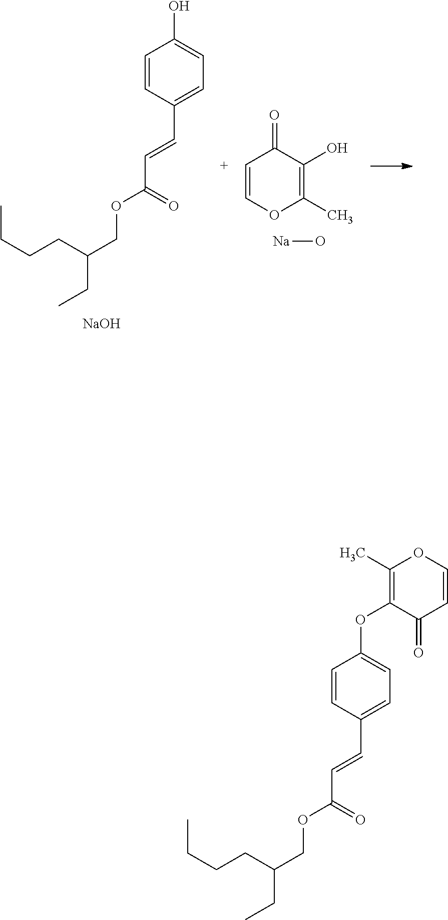

10 g (79 mmol) of 3-hydroxy-2-methyl-4-pyranonine are dissolved 100 ml of dimethylformamide in a 3-necked flask which has been flushed with argon, 22.8 g of triphenylphosphine are added, and 17.6 g of diisopropyl azodicarboxylate (87 mmol) are slowly added dropwise at 0° C. After 30 min, 2-ethylhexyl (E)-3-(4-hydroxyphenyl)acrylate (23 g, 83 mmol) dissolved in 45 ml of dimethylformamide is added dropwise. After 30 min at 0° C., the mixture is stirred at room temperature for a further 6 hours. The reaction solution is evaporated to dryness, suspended in 100 ml of ethyl acetate/hexane 1:2, filtered, and the product is precipitated out as white solid by addition of 100 ml of hexane.

### Example 5

Synthesis of (R)-5-[(R)-2-(3-benzotriazol-2-yl-4-hydroxybenzyloxy)-1-hydroxyethyl]-3,4-dihydroxy-5H-furan-2-one

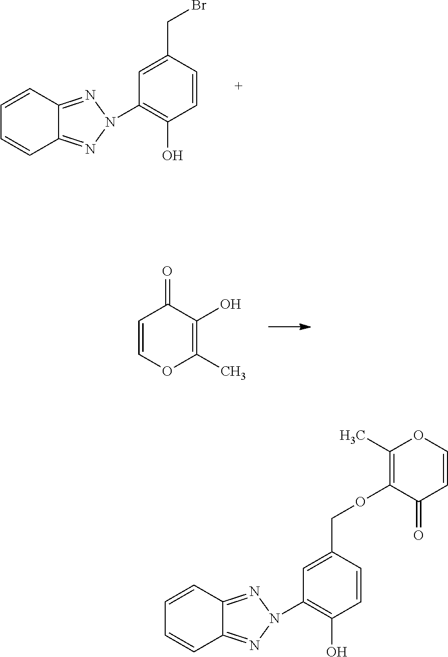

10 g of 3-hydroxy-2-methyl-4-pyranone (79 mmol) are dissolved in 250 ml of THF (tetrahydrofuran), and 3.2 g of sodium hydroxide (79 mmol) are added at 0° C. After 30 min, 2-benzotriazol-2-yl-4-bromomethylphenol (28.8 g) dissolved in 100 ml of THF is added dropwise, the mixture is stirred at 0° C. for 60 min and at 70° C. for 7 hours. After cooling to room temperature, the reaction solution is evaporated in a rotary evaporator. Re-uptake in the solvent and purification of the solution on silica gel gives (R)-5-[(R)-2-(3-benzotriazol-2-yl-4-hydroxybenzyloxy)-1-hydroxyethyl]-3,4-dihydroxy-5H-furan-2-one.

### Example 6

### Synthesis of 1,2-dimethyl-4-oxo-1,4-dihydropyridin-3-yl 2-(E)-3-(4-methoxy-phenyl)acrylate

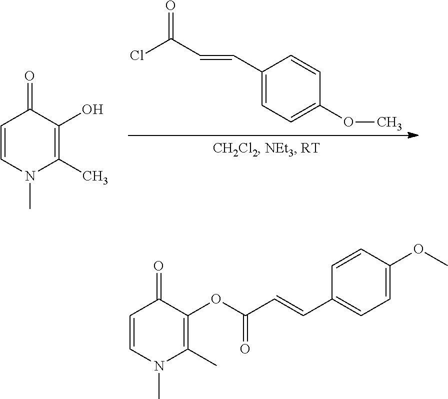

6.00 ml of triethylamine are added dropwise after 5 min to a suspension of 5.00 g (35.9 mmol) of 3-hydroxy-1,2-dimethyl-1H-pyridin-4-one in 100 ml of dichloromethane and 8.50 g (43.2 mmol) of E-4-methoxycinnamoyl chloride. The reaction mixture clarifies, and the internal temperature rises by 5° C. The orange-red reaction mixture is stirred at RT for a further 3 h. 100 ml of water are then added, and the mixture is carefully acidified using 25% hydrochloric acid. A yellow flocculent precipitate precipitates out in the process. This is separated off and dried in vacuo. After addition of a further 100 ml of water, yellow solid is again precipitated out of the mother liquor and separated off. The combined solids are stirred with 25 ml of dichloromethane for 30 min, filtered off with suction, rinsed with a little cold dichloromethane and subsequently dried at 40° C. in vacuo, giving 4.4 g (41%) of product as pale-beige powder.

¹H-NMR (400 MHz, DMSO-d₆): δ=2.49 (s, 3H), 3.78 (s, 3H), 4.07 (s, 3H), 6.69 (d, 1H, ³J=16.3 Hz), 6.98 (d, 2H, ³J=9.0 Hz), 7.35 (d, 1H, ³J=7.3 Hz), 7.71 (d, 2H, ³J=9.0 Hz), 7.89 (d, 1H, ³J=16.3 Hz), 8.56 (d, 1H, ³J=7.3 Hz).

### Example 7

### Evidence of Binding

### Determination of the Protein Affinity in the Lysine Model:

In each case, 10 mg of the substances according to Example 1 and Example 2 are weighed out into a 10 ml volumetric flask and dissolved in 5 ml of tetrahydrofuran (THF). The volumetric flask is then made up to the mark with a lysine solution in distilled water. The concentration of the lysine solution is 200 mg/I of water. The solution is stirred in the volumetric flask at 35° C. for 24 h. Samples are taken after 1 h and after 24 h. 5 μl of each of these samples are pipetted onto the start zone of an HPTLC silica gel 60 F245 plate (Merck Art. 1.05628) using a micropipette. For development of the HPTLC plate, the eluent consisting of THF/heptane in the ratio 75/25 containing 0.1 vol % of formic acid is used. After development, detection is carried out at 254 nm and 366 nm in a CAMAG ATS4, and the Rf values are determined.

Result: the substance of Example 1 has an Rf value of 0.55. After only 1 h in combination with lysine, the substance according to Example 1 has completely reacted with lysine in a Maillard reaction. The starting substance can no longer be detected. By contrast, Maillard products have formed which are detected in the start zone of the HPTLC plate. Analogous results are achieved for the substance of Example 2 (Rf 0.47), where starting substance 2 can still be detected after 1 h, but the reaction is complete after 24 h. The substance of Example 2 is then no longer detectable, while Maillard products are detected in the start zone of the HPTLC plate.

### Example 8

### Investigation in the Liquidskin Model

1 mmol of the substance according to Example 1 (2-methyl-4-oxo-4H-pyran-3-yl 2-(4-dibutylamino-2-hydroxybenzoyl)benzoate) is dissolved in 94 ml of ethylene glycol and 6 ml of water (potassium hydrogenphthalate-buffered, pH=4) with 1 mmol of DL-lysine and stirred at RT for 3 hrs. (=Liquidskin model). The L*-a*-b* values of this solution are subsequently measured using a UV/VIS spectrometer (Varian Cary-100) and compared with the starting values.

| | delta L* | delta a* | delta b* |
|---|---|---|---|
| Substance | −4.07 | −2.75 | +7.83 |
| according to | | | |
| Example 1 | | | |

The substance according to Example 1 exhibits a colour change of lysine.

### Example A

### Water-Resistant Sun-Protection Spray

| A | | | |
|---|---|---|---|
| Substance according to | 1.00 | 1.00 | 2.00 |
| Example 2 | | | |
| Diethylhexyl | | 0.50 | |
| Syringylidenemalonate, | | | |
| Caprylic/Capric | | | |
| Triglyceride | | | |
| (Oxynex ® ST Liquid) | | | |
| RonaCare ® AP | | 2.00 | |
| Ascorbyl Palmitate | | | 1.00 |
| Cyprylic/capric Triglyceride | 7.00 | 7.00 | 7.00 |
| (Miglyol 812 N) | | | |
| Butylphthalimide | 10.00 | 10.00 | 10.00 |
| isopropylphthalimide | | | |
| (Pelemol ® BIP) | | | |
| C12-15 alkyl benzoate | 10.00 | 10.00 | 10.00 |
| (Tegosoft ® TN) | | | |
| Phenethyl benzoate | 5.00 | 5.00 | 5.00 |
| (X-Tend 226) | | | |
| RonaCare ® tocopherol | 1.00 | 1.00 | 1.00 |
| acetate | | | |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, | 20.00 | 20.00 | 20.00 |
| dimethiconol | | | |
| Dow Corning 1501 Fluid | | | |
| Perfume oil (q.s.) | 0.20 | 0.20 | 0.20 |

### Example B

### Pump Hairspray

| | | | |
|---|---|---|---|
| A | | | |
| Substance according to | 1.00 | 2.00 | 4.00 |
| Example 1 | | | |
| Ethanol 96% extra pure | to 100 | to 100 | to 100 |
| PVP/VA copolymer | 6.00 | 6.00 | 6.00 |
| PVP/VA W 735 | | | |
| B | | | |
| Diethylhexyl | 0.06 | 0.25 | 0.50 |
| Syringylidenemalonate, | | | |
| Caprylic/Capric Triglyceride | | | |
| (Oxynex ® ST Liquid) | | | |
| PEG-75 Lanolin | 0.20 | 0.20 | 0.20 |
| BHT | | | |
| (Solan E - Low Dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 dimethicone | 0.50 | 0.50 | 0.50 |
| Dow Corning 193 Fluid | | | |
| 0.1% D&C Red No 33 (CI 17200) | 0.20 | 0.20 | 0.20 |
| in water | | | |
| PEG-40 Hydrogenated Castor Oil | 1.00 | 1.00 | 1.00 |
| (Cremophor RH 410) | | | |

### Example C

### W/O Emulsions

| Emulsion | A | B | C | D | E | F |
|---|---|---|---|---|---|---|
| Polyglyceryl 2- | 3 | 5 | 3 | | | |
| dipoly- | | | | | | |
| hydroxystearate | | | | | | |
| PEG-30 | | | 2 | 3 | 4 | 5 |
| dipolyhydroxy- | | | | | | |
| stearate | | | | | | |
| Sodium starch | 0.5 | 0.4 | | 0.3 | | 1 |
| octenyl succinate | | | | | | |
| Glycine | 0.3 | 0.3 | 0.5 | 0.4 | | |
| Alcohol | | 5 | 2 | 5 | 4 | |
| Magnesium | 0.2 | 0.3 | 0.3 | 0.4 | 0.5 | 0.2 |
| sulfate | | | | | | |
| C₁₂₋₁₅ alkyl | 5 | 3 | | | 5 | |
| benzoate | | | | | | |
| C₁₂₋₁₃ alkyl | | 2 | | | | |
| tartrate | | | | | | |
| Butylene glycol | 5 | | | | 3 | 3 |
| dicaprylate/ | | | | | | |
| dicaprate | | | | | | |
| Dicaprylyl ether | | | | | 2 | |
| Mineral oil | | 4 | | 6 | | 8 |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glycol | | | 1 | | 5 | 3 |
| Glycerine | 3 | 5 | 7 | 10 | 3 | 3 |
| C18-38 acid | 0.5 | | 1 | | 1 | |
| triglycerides | | | | | | |
| Titanium dioxide | 5 | 6 | 4 | | | 4 |
| Zinc oxide | 5 | | | | | |
| Bisethylhexyl- | | 3 | 3 | 2 | | |
| oxyphenol | | | | | | |
| methoxy- | | | | | | |
| phenyltriazine | | | | | | |
| Ethyl- | | 4.5 | 3 | | 3 | |
| hexyltriazone | | | | | | |
| Substance | 0.5 | | 1.0 | 1.0 | 3.0 | 1.5 |
| according | | | | | | |
| to Example 1 | | | | | | |
| Substance | 0.5 | 1.0 | | | | 1.5 |
| according | | | | | | |
| to Example 2 | | | | | | |
| Diethylhexyl- | | | 1.5 | 4 | | |
| butamido- | | | | | | |
| triazone | | | | | | |
| Butyl | 2 | 3 | 4 | | 1 | 3 |
| methoxy- | | | | | | |
| dibenzoyl- | | | | | | |
| methane | | | | | | |
| Uvinul ® A | | | | 4 | 2 | |
| Plus | | | | | | |
| Ethylhexyl | | | | | 7 | 5 |
| methoxy- | | | | | | |
| cinnamate | | | | | | |
| Taurine | 0.1 | | | 0.5 | 0.2 | |
| Vitamin E | 0.2 | 02 | | 0.3 | 0.1 | 0.5 |
| acetate | | | | | | |
| Na₂H₂EDTA | 0.1 | 0.1 | 0.2 | 0.2 | 0.2 | 0.5 |
| C8-C16 | 1 | | | | | |
| alkylpoly- | | | | | | |
| glycoside | | | | | | |
| Phenoxyethanol | 1.0 | 1.0 | 1.0 | 1.0 | 1.0 | 1.0 |
| Ethylhexyl- | 0.25 | 0.25 | 0.25 | 0.25 | 0.25 | 0.25 |
| glycerine | | | | | | |
| Sodium | q.s. | q.s. | q.s. | q.s | q.s. | q.s. |
| hydroxide | | | | | | |
| Water | to | to | to | to | to | to |
| | 100.0 | 100.0 | 100.0 | 100.0 | 100.0 | 100.0 |

### Example D

### Hair-Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ® ST | 2.000 | 2.000 | 2.000 | 2.000 | 2.000 | 2.000 |
| Substance according to | | 0.25 | 0.50 | 1.50 | 2.00 | 2.00 |
| Example 2 | | | | | | |
| Substance according to | 0.25 | | | 0.50 | | 2.00 |
| Example 1 | | | | | | |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-undecylenoyl-L-phenyl- | 0 | 0 | 0 | 0 | 1.000 | 0 |
| alanine | | | | | | |
| N-acetyl glucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosyl- | 0 | 0 | 5.000 | 0 | 0 | 0 |
| sarcosinate | | | | | | |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl | 0.200 | 0.200 | 0.200 | 0.200 | 0.200 | 0.200 |
| alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 | 3.000 | 2.000 | 2.000 | 2.000 | 2.000 | 2.000 |
| isoparaffin + laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Ascorbic acid | 0.200 | 0.200 | 0.200 | 0.200 | 0.200 | 0.200 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### Example E

### Hair-Care Formulation

Content in g of component per 100 g of formulation

| Components | G | H | I |
|---|---|---|---|
| Disodium EDTA | 0.100 | 0.100 | 0.100 |
| Oxynex ® ST | 2.000 | 2.000 | 2.000 |
| Substance according to Example 2 | 0.50 | 3.50 | 1.50 |
| Cetyl pyridinium chloride | 0.200 | 0 | 0 |
| Pitera ® | 0 | 10 | 0 |
| Ascorbyl glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerin | 7.000 | 7.000 | 7.000 |
| Titanium dioxide | 0.604 | 0.604 | 0.604 |
| Polyacrylamide + C13-14 isoparaffin + | 2.000 | 2.000 | 2.000 |
| laureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

### Example F

### O/W Emulsions

| Emulsion | A | B | C | D | E | F |
|---|---|---|---|---|---|---|
| Glyceryl stearate citrate | 2.5 | 2 | 3 | | | |
| Sorbitan stearate | 0.5 | | | 2 | 1.5 | 2 |
| Polyglyceryl-3 | | | | 2.5 | 3 | 3 |
| methylglycose distearate | | | | | | |
| Polyglyceryl-2 dipoly- | | 0.8 | | | | 0.5 |
| hydroxystearate | | | | | | |
| Cetearyl alcohol | | | | 1 | | |
| Stearyl alcohol | 2 | | | | | 2 |
| Cetyl alcohol | | 1 | | | 3 | |
| Acrylates/C₁₀₋₃₀ alkyl | | 0.2 | | | 0.1 | |
| acrylate crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylate/dicaprate | | | | | | |
| Dicaprylyl ether | | | | | 2 | |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene gycol | | | 1 | | 5 | 3 |
| Glycerine | 3 | 5 | 7 | 10 | 3 | 3 |
| C18-C38 acid triglycerides | 0.5 | | 1 | | 1 | |
| Titanium dioxide | 5 | | | 2 | | |
| 2,2′-Methylen-bis-(6-(2H- | 2.5 | | | | | |
| benzotriazol-2-yl)-(1,1,3,3- | | | | | | |
| tetramethylbutyl)phenol) | | | | | | |
| 2,4,6-Tris-(biphenyl)- | | 2 | | | | |
| 1,3,5-triazine | | | | | | |
| C8-C16 alkylpolyglycoside | 1 | 0.6 | | | | |
| UVASorb ® K2A | | | 2 | | | |
| Uvinul ® A Plus | 2 | | | | | 1 |
| Homosalate | | 5 | | 1 | | |
| Phenylbenzimidazole | | | 2 | | | 1 |
| sulfonic acid | | | | | | |
| Benzophenone-3 | 2 | | | | 2 | |
| Octyl salicylate | 5 | 5 | | 2 | | |
| Octocrylene | 2 | | | | 3 | 1 |
| Substance according to | 1.0 | | | | | 0.5 |
| Example 1 | | | | | | |
| Substance according to | | 1.0 | | | | 0.5 |
| Example 2 | | | | | | |
| Substance according to | | | 1.0 | | | 0.5 |
| Example 3 | | | | | | |
| Substance according to | | | | 1.0 | | 0.5 |
| Example 4 | | | | | | |
| Substance according to | | | | | 1.0 | 0.5 |
| Example 5 or 6 | | | | | | |
| Bis-ethylhexyloxyphenol | | 3 | 2 | 1 | | |
| methoxyphenyltriazine | | | | | | |
| Parsol ® SLX | | | 3 | | | |
| Dihydroxyacetate | | | | | 4 | |
| Taurine | 0.1 | | | 0.5 | 0.2 | |
| 8-Hexadecene-1,16- | | 0.2 | | | | |
| dicarboxylic acid | | | | | | |
| Vitamin E acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to 100.0 | to | to | to | to | to |
| | | 100.0 | 100.0 | 100.0 | 100.0 | 100.0 |

### Example G

### O/W Emulsions

| Emulsion | G | H | I | K | L | M |
|---|---|---|---|---|---|---|
| Ceteareth-20 | 1 | 1.5 | 1 | | | |
| Sorbitan stearate | | | 0.5 | | 0.5 | |
| Glyceryl stearate | | | | 1 | 1 | 1.5 |
| SE | | | | | | |
| Emulgade F ® | | | | 2.5 | 2.5 | 3 |
| Cetearyl alcohol | | | | 1 | | |
| Stearyl alcohol | | | | | 1.5 | |
| Cetyl alcohol | | | 0.5 | | | 2 |
| Acrylates/C₁₀₋₃₀ | 0.2 | 0.4 | 0.3 | 0.1 | | |
| alkyl acrylate | | | | | | |
| crosspolymer | | | | | | |
| Carbomer | | | | | 0.3 | |
| Xanthan Gum | | | | 0.4 | | 0.4 |
| C₁₂₋₁₅ alkyl | 5 | 3 | | | 5 | |
| benzoate | | | | | | |
| 2-Phenyl | | 2 | | | | |
| benzoate | | | | | | |
| Butylene glycol | 5 | | | | 3 | 2 |
| dicaprylate/ | | | | | | |
| dicaprate | | | | | | |
| Dicaprylyl ether | | | | | 2 | |
| Diethylhexyl | 2 | | | | | |
| naphthalate | | | | | | |
| Dicapryl caprate | | 2 | | | 2 | 2 |
| Cyclomethicone | 5 | | 5 | 10 | | |
| Isohexadecane | | | | 5 | | |
| Mineral oil | | 1 | | | | |
| Propylene glycol | | | 4 | | | |
| Glycerine | 5 | 7 | 3 | 5 | 6 | 8 |
| C18-38 acid | 0.5 | | 1 | | 1 | |
| triglycerides | | | | | | |
| Titanium dioxide | 5 | | 3 | 2 | | |
| NeoHeliopan ® | | 2 | | | 1 | 1 |
| AP | | | | | | |
| Phenyl- | 1 | | | 1 | 2 | 1 |
| benzimidazole | | | | | | |
| sulfonic acid | | | | | | |
| Ethylhexyl | 5 | | 4 | 4 | | |
| methoxy- | | | | | | |
| cinnamate | | | | | | |
| Ethyl- | | 2 | | 1 | | |
| hexyltriazone | | | | | | |
| Diethyl- | 1 | | | | | |
| hexylbutamido- | | | | | | |
| triazane | | | | | | |
| Butyl | 2.5 | | 2 | 2 | | 1 |
| methoxy- | | | | | | |
| dibenzoyl- | | | | | | |
| methane | | | | | | |
| Bis- | 2 | | | | | |
| ethyl- | | | | | | |
| hexyloxyphenol | | | | | | |
| methoxy- | | | | | | |
| phenyltriazine | | | | | | |
| 4- | 3 | | | | | |
| Methyl- | | | | | | |
| benzylidene | | | | | | |
| camphor | | | | | | |
| Parsol ® SLX | | | | | 2 | |
| Substance | 1.0 | | 0.5 | 1.0 | 2.5 | |
| according | | | | | | |
| to Example 2 | | | | | | |
| Substance | | 1.0 | 0.5 | 1.0 | | 2.5 |
| according | | | | | | |
| to Example 1 | | | | | | |
| Creatinine | 0.1 | 0.01 | 0.05 | | | |
| Creatine | 0.5 | 0.2 | 0.1 | | | |
| Licorice extract/ | | | | 0.5 | | |
| licochalcone | | | | | | |
| Vitamin E acetate | 0.2 | | | 0.5 | 0.5 | 0.5 |
| Tapioca starch | | 3 | | | 2 | |
| Na₂H₂EDTA | 0.1 | | 0.2 | | | 0.5 |
| Perfume, | q.s. | q.s. | q.s. | q.s. | q.s. | q.s. |
| preservatives | | | | | | |
| Dyes, etc. | q.s. | q.s. | q.s. | q.s. | q.s. | q.s. |
| Sodium | q.s. | q.s. | q.s. | q.s. | q.s. | q.s. |
| hydroxide | | | | | | |
| Water | to 100.0 | to | to | to | to | to |
| | | 100.0 | 100.0 | 100.0 | 100.0 | 100.0 |

### Example H

### O/W Emulsions

| Emulsion | N | O | P | Q | R | S |
|---|---|---|---|---|---|---|
| Glyceryl | | 2 | | 2 | | |
| stearate SE | | | | | | |
| Glyceryl stearate | 2 | | 2 | | | |
| PEG-40 stearate | | | 2 | | 1 | |
| PEG-10 stearate | | | | 2.5 | 1 | |
| Ceteareth-20 | | | | | | 2.6 |
| Sodium cetyl | | | | | 2 | |
| phosphate | | | | | | |
| Glyceryl stearate, | | | | | | 5.4 |
| ceteareth-12, | | | | | | |
| ceteareth-20, | | | | | | |
| cetearyl alcohol, | | | | | | |
| cetyl palmitate | | | | | | |
| Stearic acid | 3 | 2 | | | 2 | |
| Stearyl alcohol | | 2 | 2 | | | |
| Stearyl alcohol | 0.5 | | 2 | | | |
| Cetyl alcohol | 3 | | | 2 | | |
| Acrylates/C₁₀₋₃₀ | | | 0.2 | | 0.4 | |
| alkyl acrylate | | | | | | |
| crosspolymer | | | | | | |
| Carbomer | | 0.3 | | 0.3 | 0.3 | |
| Xanthan Gum | | 0.3 | 0.4 | | | |
| C₁₂₋₁₅ alkyl | 5 | | | | 5 | 3 |
| benzoate | | | | | | |
| 2-Phenyl | 5 | | | | | |
| benzoate | | | | | | |
| Butylene glycol | | 5 | | 4 | | 3 |
| dicaprylate/ | | | | | | |
| dicaprate | | | | | | |
| Dicaprylyl ether | | 2 | | | 3 | |
| Diethylhexyl | 3 | | | | | |
| naphthalate | | | | | | |
| Cyclomethicone | 2 | | 10 | 2 | | |
| Isohexadecane | | | | 2 | 3 | |
| Mineral oil | | | | | 3 | |
| Propanediol | | 3 | | 5 | | |
| Glycerine | 3 | 5 | 10 | 7 | 4 | 5 |
| Titanium dioxide | 2 | 4 | | | | |
| Zinc oxide | | | | | 2 | |
| Drometrizole | | | | | 3 | |
| Trisiloxane | | | | | | |
| Ethylhexyl | | 6 | 5 | | | |
| methoxy- | | | | | | |
| cinnamate | | | | | | |
| Phenyl- | | 0.5 | 2 | | 1 | |
| benzimidazole | | | | | | |
| sulfonic acid | | | | | | |
| Homosalate | 5 | | | 7 | | |
| Butyl | | 3 | | | | |
| methoxy- | | | | | | |
| dibenzoyl- | | | | | | |
| methane | | | | | | |
| Bis- | | 2 | 3 | | | |
| ethyl- | | | | | | |
| hexyloxyphenol | | | | | | |
| methoxy- | | | | | | |
| phenyltriazine | | | | | | |
| Octyl salicylate | | | | 5 | | |
| Octocrylene | | | | | 3 | |
| Substance | 0.25 | 1.5 | 0.5 | 2.5 | 1.0 | 5.0 |
| according | | | | | | |
| to Example 2 | | | | | | |
| Parsol ® SLX | 4 | | | | | 5 |
| PVP hexadecene | 0.5 | | 1 | | 0.8 | |
| copolymer | | | | | | |
| Coenzyme Q 10 | 0.2 | 0.02 | | 0.3 | | |
| Vitamin E | 0.2 | | 0.3 | | 0.8 | 0.5 |
| acetate | | | | | | |
| Na₂H₂EDTA | 0.1 | | | | | 0.5 |
| Perfume, | q.s. | q.s. | q.s. | q.s. | q.s. | q.s. |
| preservatives | | | | | | |
| Dyes, etc. | q.s. | q.s. | q.s. | q.s. | q.s. | q.s. |
| Sodium | q.s. | q.s. | q.s. | q.s. | q.s. | q.s. |
| hydroxide | | | | | | |
| Water | to | to | to | to | to | to |
| | 100.0 | 100.0 | 100.0 | 100.0 | 100.0 | 100.0 |

### Example I

### Hydrodisperions (Lotions and Sprays)

| | A | B | C | D | E | F |
|---|---|---|---|---|---|---|
| Glyceryl stearate | | 0.40 | | | | |
| citrate | | | | | | |
| Cetyl alcohol | | | | | 2.00 | |
| Sodium carbomer | | | | | 0.30 | |
| Acrylates/C₁₀₋₃₀ | 0.30 | | 0.30 | 0.40 | 0.10 | 0.10 |
| alkyl acrylate | | | | | | |
| crosspolymer | | | | | | |
| Cetsareth-20 | | | 1.00 | | | |
| Xanthan gum | | | | 0.15 | | 0.50 |
| Dimethicone/ | | | | 5.00 | | 3.00 |
| vinyl | | | | | | |
| dimethicone | | | | | | |
| crosspolymer | | | | | | |
| UVASorb ® | | | | | 3.50 | |
| K2A | | | | | | |
| Uvinul ® A Plus | 0.25 | | | 0.50 | 2.00 | 1.50 |
| Butyl | 1.20 | | 3.50 | | | |
| methoxy- | | | | | | |
| dibenzoyl- | | | | | | |
| methane | | | | | | |
| Bis- | 2.00 | 2.00 | | 0.25 | | |
| ethyl- | | | | | | |
| hexyloxyphenol | | | | | | |
| methoxyphenyl | | | | | | |
| triazine | | | | | | |
| Terephthalidene | | | | | | 0.50 |
| dicamphor | | | | | | |
| sulfonic acid | | | | | | |
| Disodium phenyl | | | | | | 1.00 |
| dibenzimidazole | | | | | | |
| tetrasulfonate | | | | | | |
| Phenyl- | | | 2.00 | | | |
| benzimidazole | | | | | | |
| sulfonic acid | | | | | | |
| Ethylhexyl - | 5.00 | | 7.00 | | 5.00 | 8.00 |
| methoxy- | | | | | | |
| cinnamate | | | | | | |
| Diethylhexyl | | | 2.00 | 2.00 | | |
| butamido triazone | | | | | | |
| Ethylhexyl - | 4.00 | 3.00 | | | 4.00 | |
| triazone | | | | | | |
| Octocrylene | | | | 10.00 | | 2.50 |
| Substance | 0.25 | | 0.5 | | 1.0 | 2.0 |
| according | | | | | | |
| to Example | | | | | | |
| 2, 3, 4, 5 or 6 | | | | | | |
| Substance | | 0.5 | | 1.0 | | 1.0 |
| according | | | | | | |
| to Example 1 | | | | | | |
| C₁₂₋₁₅ alkyl | 2.00 | | 2.50 | | | |
| benzoate | | | | | | |
| Phenethyl | 4.00 | | | 7.50 | | 5.00 |
| benzoate | | | | | | |
| C₁₈₋₃₆ triglyceride | | | 1.00 | | | |
| fatty acid | | | | | | |
| Butylene glycol | | | | | 6.00 | |
| dicaprylate/ | | | | | | |
| dicaprate | | | | | | |
| Dicaprylyl | | 3.00 | | | | |
| carbonate | | | | | | |
| Dicaprylyl ether | | 2.00 | | | | |
| Cyclomethicone | | | | 1.50 | | |
| Lanolin | | | | | 0.35 | |
| PVP hexadecene | 0.50 | | 0.50 | | 0.50 | 1.00 |
| copolymer | | | | | | |
| Ethyl - | | 0.75 | | 1.00 | | 0.50 |
| hexyloxyglycerine | | | | | | |
| Glycerine | 10.00 | 5.00 | 5.00 | | 5.00 | 15.00 |
| Butylene glycol | | 7.00 | | | | |
| Glycine soya | | | | 1.00 | | |
| Vitamin E acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Idopropynyl butyl- | 0.20 | 0.10 | | | | 0.15 |
| carbamate | | | | | | |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perfume, dyes | q.s. | q.s. | q.s. | qs. | q.s. | q.s. |
| Water | to | to | to | to | to | to |
| | 100 | 100 | 100 | 100 | 100 | 100 |
| Neutralisers | qs | qs | qs | qs | qs | qs |
| (sodium | | | | | | |
| hydroxide, | | | | | | |
| potassium | | | | | | |
| hydroxide) | | | | | | |

### Example J

### Aqueous and Aqueous/Alcoholic Formulations

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C10-30 alkyl | | | | 0.3 | 0.6 | |
| acrylate crosspolymer | | | | | | |
| Cocamidopropylbetaine | | | 0.3 | | | |
| UVASorb ® K2A | | | | | 2 | |
| Uvinul ® APlus | 5 | | | | | |
| Butyl | 0.5 | | | 3 | | |
| methoxydibenzoyl- | | | | | | |
| methane | | | | | | |
| Disodium phenyl | | 2 | 1 | | | |
| dibenzimidazole | | | | | | |
| tetrasulfonate | | | | | | |
| Phenylbenzimidazole | | 5 | 3 | | 2 | 4 |
| sulfonic acid | | | | | | |
| Ethylhexyl methoxy- | 10 | | | | 3 | |
| cinnamate | | | | | | |
| Diethylhexyl butamido | | | | | 3 | |
| triazone | | | | | | |
| Ethylhexyl triazone | | | | | 2 | |
| Octocrylene | | | | 5 | | |
| Substance according | 2.5 | 0.75 | 1.5 | 3.0 | 3.5 | 4.0 |
| to Example 2 | | | | | | |
| C₁₂₋₁₅ alkyl benzoate | | | | 3 | | |
| C18-36 triglyceride | | | | 1 | | |
| fatty acid | | | | | | |
| Butylene glycol | 2 | | | | | |
| dicaprylate/dicaprate | | | | | | |
| C12-13 alkyl tartrate | | | | | 5 | |
| Cyclomethicone | 4 | | | 2 | | |
| Insect Repellent ® | | | | 5 | | |
| 3535 | | | | | | |
| Dimethicone | | | | | 3 | |
| PVP hexadecene | | 0.5 | | 1 | | 0.5 |
| copolymer | | | | | | |
| Ethylhexylglycerine | | 0.5 | | | | |
| Glycerine | 5 | 7 | 3 | 8 | | S |
| Butylene glycol | | | 5 | | 5 | |
| Metylpropanediol | | | | 4 | | |
| Vitamin E acetate | | 0.3 | 0.2 | 0.5 | | |
| Panthenol | 0_{:}5 | | 0.2 | | | 0.3 |
| Creatinine | | | 0.01 | | 0.02 | |
| Creatine | | | 0.1 | | 0.2 | |
| PEG-40 hydrogenated | | 0.5 | 0.3 | | | 0.5 |
| castor oil | | | | | | |
| Trisodium EDTA | 0.3 | 0.2 | 0.2 | 0.2 | 0.2 | 0.5 |
| Methylparaben | q.s. | q.s. | q.s. | q.s. | q.s. | q.s. |
| Sodium hydroxide | q.s. | q.s. | q.s. | q.s. | q.s. | q.s. |
| Perfume, dyes | q.s. | q.s. | q.s. | q.s. | q.s. | q.s. |
| Water | to 100 | to 100 | to 100 | to | to 100 | to |
| | | | | 100 | | 100 |

### Example K

### Cosmetic Foams

| Emulsion | A | B | C |
|---|---|---|---|
| Stearic acid | 2 | 2 | |
| Palmitic acid | | | 1.5 |
| Cetyl alcohol | 2.5 | 2 | |
| Stearyl alcohol | | | 3 |
| PEG-100 stearate | | | 3.5 |
| PEG-40 stearate | | 2 | |
| PEG-20 stearate | 3 | | |
| Sorbitan stearate | | 0.8 | |
| C₁₂₋₁₅ alkyl benzoate | 5 | | |
| C₁₂₋₁₃ alkyl tartrate | | | 7 |
| Butylene glycol | | 6 | |
| dicaprylate/dicaprate | | | |
| Dicaprylyl ether | | | 2 |
| Cyclomethicone | | 2 | 3 |
| Butylene glycol | 1 | | |
| Isohexadecane | 2 | | |
| Methylpropanediol | | | |
| Propylene glycol | | | 5 |
| Glycerine | 5 | 7 | |
| UVASorb ® K2A | | | 2 |
| Uvinul ® A Plus | 2 | 3 | |
| Substance according to | 0.5 | 1.0 | 1.5 |
| Example 2 | | | |
| Parsol SLX ® | | 3 | |
| Homosalate | | 5 | |
| Phenylbenzimidazole sulfonic | | 2 | 2 |
| acid | | | |
| Benzophenone-3 | 2 | | |
| Octyl salicylate | | 5 | |
| Octocrylene | 2 | | |
| Bis-ethylhexyloxyphenol | | 3 | |
| methoxyphenyltriazine | | | |
| 2,2′-Methylene-bis-(6-(2H- | | | 8 |
| benzotriazol-2-yl)-4-(1,1,3,3- | | | |
| tetramethylbutyl)phenol) | | | |
| 2,4,6-Tris-(biphenyl)-1,3 | 5 | | 4 |
| 5-triazine | | | |
| C8-C16 alkylpolyglycosides | 1 | | |
| Vitamin E acetate | 0.6 | 0.5 | 0.2 |
| Creatine/Creatinine | | | 0.5 |
| Na₂H₂EDTA | 0.50 | | |
| Phenoxyethanol | 1.0 | 1.0 | 1.0 |
| Ethylhexylglycerine | 0.5 | 0.5 | 0.5 |
| Sodium hydroxide | q.s. | q.s. | q.s. |
| Water | to 100.0 | to 100.0 | to 100.0 |

### Example K Continuation

### Cosmetic Foams

| Emulsion | D | E | F | G |
|---|---|---|---|---|
| Stearic acid | 2 | | | |
| Palmitic acid | | | 3 | 3 |
| Cetyl alcohol | 2 | 2 | | |
| Cetylstearyl alcohol | | | 2 | 2 |
| Stearyl alcohol | | | | |
| PEG-100 stearate | | 4 | | |
| PEG-40 stearate | 2 | | | |
| PEG-20 stearate | | | 3 | 3 |
| Sorbitan stearate | 0.8 | | | |
| Tridecyl Trimellitate | | 5 | | |
| C₁₂₋₁₅ alkyl benzoate | | | 3 | 3 |
| Butylene glycol | 8 | | | |
| dicaprylate/dicaprate | | | | |
| Octyldodecanol | | 2 | | |
| Cocoglycerides | | | | 2 |
| Dicaprylyl ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerine | 5 | | 6 | 6 |
| NeoHeliopan ® AP | | 2 | | |
| Phenylbenzimidazole | 1 | | | 1 |
| sulfonic acid | | | | |
| Substance according | 0.75 | | 1.5 | |
| to Example 2 | | | | |
| Substance according | | 0.75 | 1.5 | 3.0 |
| to Example 1 | | | | |
| Ethylhexyl methoxy- | 5 | | 4 | 4 |
| cinnamate | | | | |
| Ethylhexyltriazone | | 2 | | 1 |
| Eusolex T-AVO ® | 2 | | | |
| Diethylhexylbutamido- | 1 | | | |
| triazone | | | | |
| Butyl methoxydibenzoyl- | 2.5 | | 2 | 2 |
| methane | | | | |
| Bis-ethylhexyloxyphenol | 2 | | | |
| methoxyphenyltriazine | | | | |
| Vitamin E acetate | 0.2 | | 0.3 | 0.3 |
| Na₂H₂EDTA | | | | |
| Perfume, preservatives | | | | |
| Dyes, etc. | | | | |
| Sodium hydroxide | | q-s. | q.s. | |
| Triethanolamine | q.s. | | | q.s. |
| Water | to 100.0 | to 100.0 | to 100.0 | to 100.0 |

### Example L

### Hair Rinse

| | Percent by weight [%] |
|---|---|
| Cetearyl Alcohol | 10 |
| Sunflowerseedamidopropyl Ethyldimonium | 0.5 |
| Ethosulfate | |
| Ceteareth-20 | 3.0 |
| Panthenol | 0.4 |
| Phenyl Trimethicone | 0.3 |
| Hydroxypropyl Guar Hydroxypropyltrimonium | 0.8 |
| Chloride | |
| Substance according to Example 2 | 1.0 |
| Passiflora Incarnata Seed Oil | 0.2 |
| Basic red 51 | 0.1 |
| Basic red 76 | 0.2 |
| Perfume | 1.0 |
| Preservatives | q.s. |
| Citric Acid/Sodium Hydroxide | q.s. to pH 5.5 |
| Aqua | to 100 |

### Example M

### Hair Rinse

| | Percent by weight [%] |
|---|---|
| Cetearyl Alcohol | 5.0 |
| Cetrimonium Chloride | 1.0 |
| Polysilicone-15 | 0.5 |
| Panthenol | 0.4 |
| Dimethicone | 0.8 |
| Hydroxypropyl Guar Hydroxypropyltrimonium | 1.0 |
| Chloride | |
| Substance according to Example 1 | 0.5 |
| Substance according to Example 2 | 0.5 |
| Hydrogenated Grapeseed Oil | 1.5 |
| Tiliroside | 0.05 |
| Avocado Extract | 0.5 |
| Perfume | 1.0 |
| Preservatives | q.s. |
| Citric Acid/Sodium Hydroxide | q.s. to pH 5.5 |
| Aqua | to 100 |

### Example N

### Hair Foam

| | Percent by weight [%] |
|---|---|
| Quaternium-80 | 0.2 |
| Polyquaternium-11 | 0.7 |
| PEG-60 Hydrogenated Castor Oil | 0.5 |
| Phenoxyethanol | 1.0 |
| RonaCare ® AP | 0.25. |
| Substance according to Example 1, 2, 3, 4, 5 | 0.3 |
| or 6 | |
| Passiflora Incarnata Seed Oil | 0.2 |
| Citric Acid/Sodium Hydroxide | q.s. to pH 4.5 |
| Aqua | to 100 |

The formulation can be employed as leave-on or rinse-off formulation and is packaged in a pressure container as aerosol in the ratio formulation/propellant gas=90/10. Propellant gases which can be employed are, for example, propane or butane or mixtures thereof.

### Example O

### Shampoo

| | Percent by weight [%] |
|---|---|
| Sodium Laureth Sulfate | 5.0 |
| Cocamidopropy Betaine | 5.0 |
| Lauroyl Glutamic Acid | 3.0 |
| Decyl Glucoside | 5.0 |
| Polyquaternium-10 | 0.5 |
| PEG-3 Distearate | 0.8 |
| Substance according to Example 2 | 0.5 |
| Evening primrose oil | 0.3 |
| Basic Red 51 | 0.1 |
| Ubiquinone | 0.1 |
| Decane-1,2-diol | 0.5 |
| Perfume | 1.0 |
| Preservatives | q.s. |
| Sodium Chloride | 0.8 |
| Citric Acid/Sodium Hydroxide | q.s. to pH 5.5 |
| Aqua | to 100 |

### Example P

### Shampoo

| | Percent by weight [%] |
|---|---|
| Palm Kernel/Coco Glucoside | 5.0 |
| Cocamidopropyl Betaine | 6.0 |
| Sodium Laureth Sulfate | 4.0 |
| Guar Hydroxypropyl Trimonium Chloride | 0.5 |
| Benzyl Alcohol | 0.5 |
| Hemp seed oil | 0.5 |
| Substance according to Example 1, 2, 3, 4, 5 | 0.5 |
| or 6 | |
| Perfume | 1.0 |
| Preservatives | q.s. |
| Sodium Chloride | 0.8 |
| Lactid Acid/Sodium Hydroxide | q.s. to pH 5.0 |
| Aqua | to 100 |

### Example Q

### Shampoo

| | Percent by weight [%] |
|---|---|
| Sodium Lauryl Glucose Carboxylate | 5.0 |
| Palm Kernel/Coco Glucoside | 5.0 |
| Cocamidopropyl Betaine | 5.0 |
| Polyquaternium-7 | 0.2 |
| Substance according to Example 1, 2, 3, 4, 5 | 0.5 |
| or 6 | |
| Benzyl Alcohol | 0.5 |
| Passiflora Incarnata Seed Oil | 0.5 |
| PEG-60 Hydrogenated Castor Oil | 0.5 |
| PEG-18 Glyceryl Cocoate/Oleate | 1.0 |
| Perfume | 1.0 |
| Preservatives | q.s. |
| Sodium Chloride | 0.8 |
| Lactid Acid/Sodium Hydroxide | q.s. to pH 6.0 |
| Aqua | to 100 |

### Example R

### Hair Dyeing Recipes

| | 1 | 2 | 3 | 4 | 5 | 6 | 7 |
|---|---|---|---|---|---|---|---|
| Benzyl Alcohol | 2.5 | | | | | | |
| Propylene Carbonate | 10 | | | | | | |
| Ethanol | 5.0 | | | | | | |
| Hydroxyethylcellulose | 2.0 | | | | | | |
| Pirenoxine | 2.0 | | | | | | 2.0 |
| SodiumCAS | | | | | | | |
| 51410-30-1 | | | | | | | |
| Tramsanguin | | 1.0 | | | | 1.0 | |
| CAS 34083-17-5 | | | | | | | |
| Cinnabarin | | | 1.0 | | | | |
| CAS 606-59-7 | | | | | | | |
| Cinnabaric acid | | | | 1.0 | | | |
| CAS 146-90-7 | | | | | | | |
| Resorcinol Blue | | | | | 1.0 | | |
| CAS 71939-12-3 | | | | | | | |
| Substance according | 1.0 | 1.0 | 2.0 | 1.0 | 1.0 | 0.5 | |
| to Example 1, 2, 3, | | | | | | | |
| 4, 5 or 6 | | | | | | | |
| Perfume | 1.0 | 1.0 | 1.0 | 1.0 | 1.0 | 1.0 | 1.0 |
| Preservatives | q.s. | q.s. | q.s. | q.s. | q.s. | q.s. | q.s. |
| Citric Acid | q.s. | q.s. | q.s. | q.s. | q.s. | q.s. | q.s. |
| | to | to | to | to | to | to | to |
| | pH | pH | pH | pH | pH | pH | pH |
| | 5.5 | 5.5 | 5.5 | 5.5 | 5.5 | 5.5 | 5.5 |
| Aqua | to | to | to | to | to | to | to |
| | 100 | 100 | 100 | 100 | 100 | 100 | 100 |

1. A method comprising contacting a compound of the formula I, to a protein, 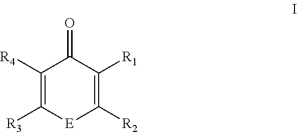
where
E denotes NR₅ or O,
R₁ denotes a UV chromophore, which is bonded via an O atom and renders the compound of the formula I capable of absorbing UV radiation in the range from 400 to 200 nm,
R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms and/or salts, tautomers, stereoisomers and/or solvates thereof, including mixtures thereof in all ratios, as a protein-adhesive active compound.

2. A method comprising contacting skin, hair and/or nails with a compound of the formula I, 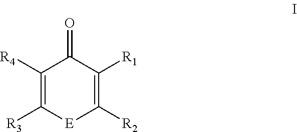
where
E denotes NR₅ or O,
R₁ denotes a UV chromophore, which is bonded via an O atom and renders the compound of the formula I capable of absorbing UV radiation in the range from 400 to 200 nm,
R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms and/or salts, tautomers, stereoisomers and/or solvates thereof, including mixtures thereof in all ratios, for protection of the skin, of the hair and/or of the nails against UV-induced damage.

3. Method according to claim 1, where, in formula I,
R₁ is a substituent of the formula II, III, IV, V, VI, VII, VIII, IX, X, XI, XII, XIII, XIV or XV, 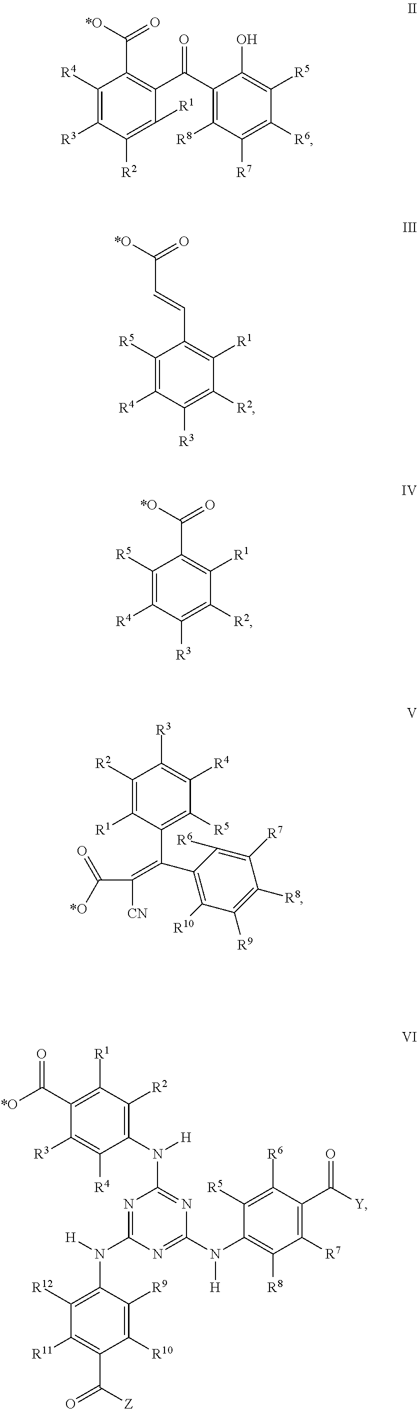 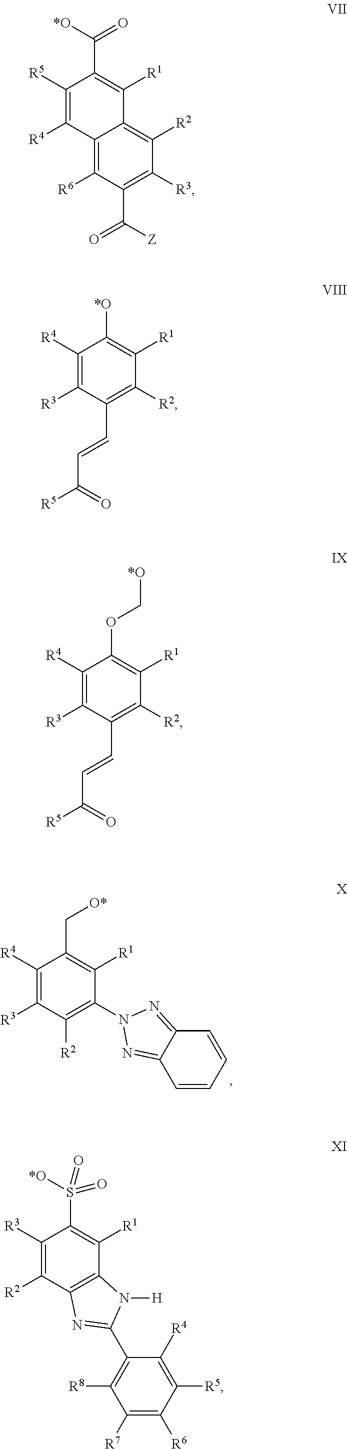 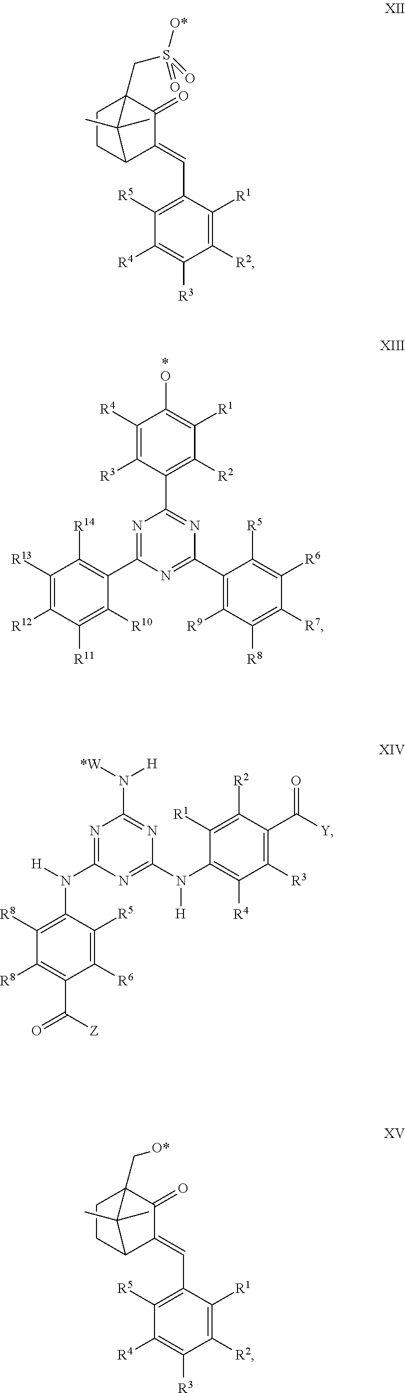
where
R¹ to R¹⁴ each, independently of one another, denote —H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or 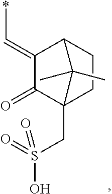 [SMILES: */C=C1/C(=O)C2(CS(=O)(=O)O)CCC1C2(C)C]
n is an integer from 1 to 25,
X is the counterion to the cations [NHA₂]⁺ and [NA₃]⁺ or the anion [SO₃]⁻ and
Y and Z each, independently of one another, denote the structural unit I-1 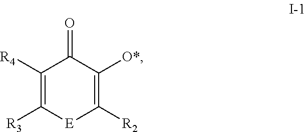
A, hydroxyl, —OA or —NH—C(CH₃)₃, where R₂, R₃, R₄ and E have a meaning indicated in claim 1,
W denotes —(CH₂)ₘ—O or —(CH₂)ₒ—C(O)—O and
m and o each, independently of one another, denote an integer from 1 to 4,
with the proviso that at least one substituent of the substituents R¹ to R¹⁴ in the formulae II, III, IV, VII, X, XII and XV denotes
OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or

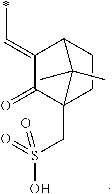 [SMILES: */C=C1/C(=O)C2(CS(=O)(=O)O)CCC1C2(C)C]

4. Compounds of the formula I 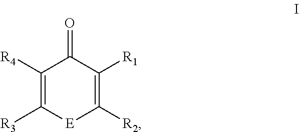
where
E denotes NR₅ or O,
R₁ is a substituent of the formula II, III, IV, V, VI, VII, VIII, IX, X, XI, XII, XIII, XIV or XV, 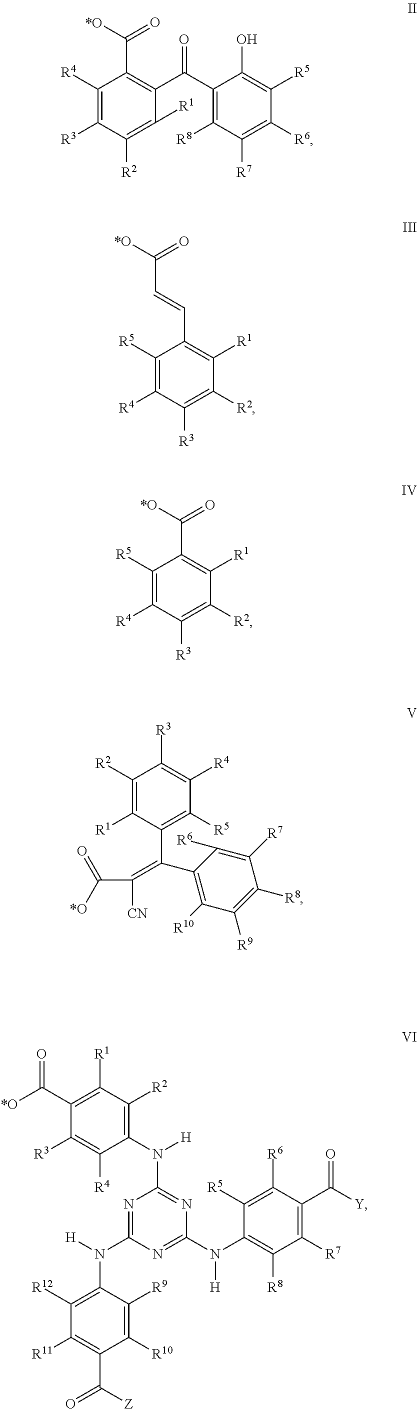 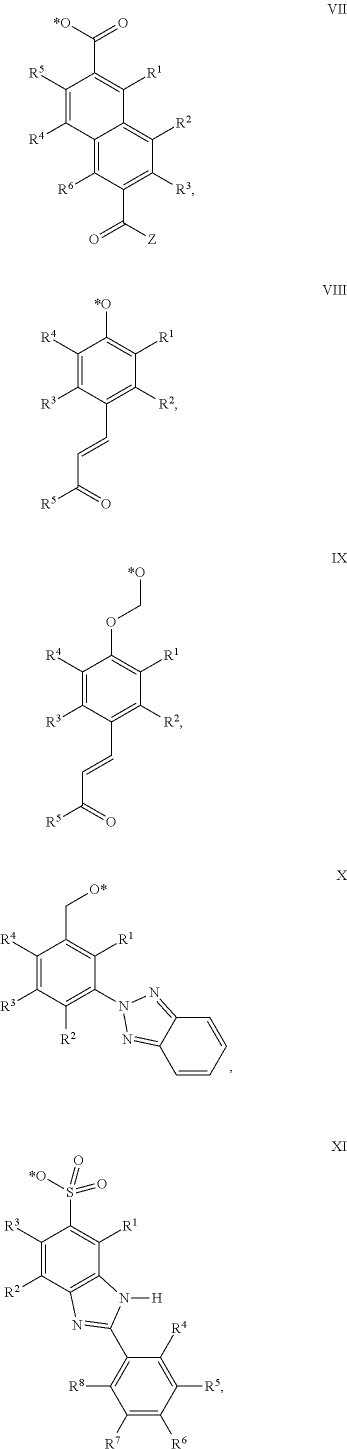 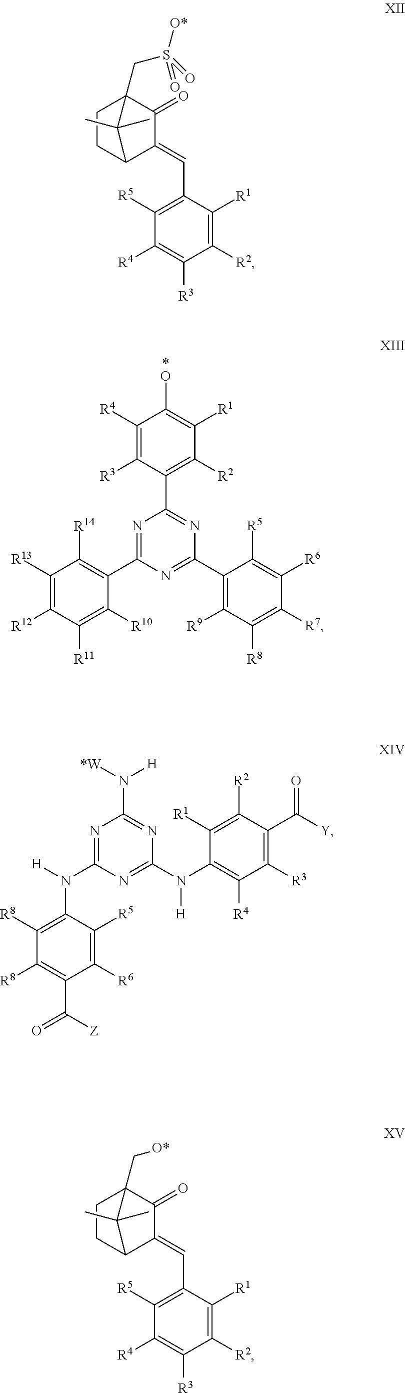
where
R¹ to R¹⁴ each, independently of one another, denote —H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or 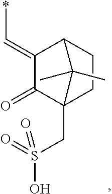 [SMILES: */C=C1/C(=O)C2(CS(=O)(=O)O)CCC1C2(C)C]
n is an integer from 1 to 25,
X is the counterion to the cations [NHA₂]⁺ and [NA₃]⁺ or the anion [SO₃]⁻ and
Y and Z each, independently of one another, denote the structural unit I-1 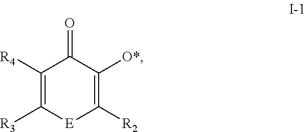
A, hydroxyl, —OA or —NH—C(CH₃)₃,
W denotes —(CH₂)ₘ—O or —(CH₂)ₒ—C(O)—O and
m and o each, independently of one another, denote an integer from 1 to 4,
with the proviso that at least one substituent of the substituents R¹ to R¹⁴ in the formulae II, III, IV, VII, X, XII and XV denotes
OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl- or 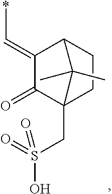 [SMILES: */C=C1/C(=O)C2(CS(=O)(=O)O)CCC1C2(C)C]
R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group,
R₅ denotes A and
A denotes alkyl having 1 to 20 C atoms,
where the compounds 2-methyl-4-oxo-4H-pyran-3-yl (E)-3-(4-hydroxy-phenyl)-acrylate or 2-methyl-4-oxo-4H-pyran-3-yl 2-hydroxybenzoate are excluded.

5. Compounds according to claim 4, characterised in that, in formula II, R⁶ preferably —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula III, R³ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula IV, R³ or R⁵ denote H, —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl, where at least one substituent R³ or R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula IV, R² denotes 2H-benzotriazol-2-yl and R¹ or R³ each, independently, preferably denote H, —OH, —OA, -A, —NH₂, —NHA, —NA₂, —NH—(CH₂—CH₂—O)ₙ—H or —N[(CH₂—CH₂—O)ₙ—H]₂ and/or
the substituents R², R⁴, R⁷ and R⁹ in formula V are H and the substituents R¹, R³, R⁵, R⁶, R⁸ and R¹⁰ are each, independently of one another, H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and/or
the substituents R², R⁴, R⁵, R⁸, R⁹ and R¹² of the formula VI are each, independently of one another, H or OH and the substituents R¹, R³, R⁶, R⁷, R¹⁰ and R¹¹ are each, independently of one another, H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and Y and Z are hydroxyl, —OA or —NH—C(CH₃)₃ and/or
the substituents R² and R⁴ of the formula VII are H and at least one of the substituents R¹, R³, R⁵ and R⁶ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula VIII, R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula IX, R⁵ denotes —OH, —OA, -A, —NH₂, —NHA, —NA₂, —NH—(CH₂—CH₂—O)ₙ—H, —N[(CH₂—CH₂—O)ₙ—H]₂, SO₃H, SO₃X or 2H-benzotriazol-2-yl and/or
in formula X, R¹ and/or R² denote —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and/or
in formula XI, R⁶ denotes H, —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X or 2H-benzotriazol-2-yl and/or
in formula XII, R³ denotes —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or 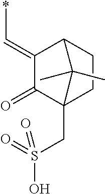 [SMILES: */C=C1/C(=O)C2(CS(=O)(=O)O)CCC1C2(C)C]
and/or
in formula XIII, R⁵, R⁷, R¹² and R¹⁴ each, independently of one another, preferably denote —OH, —OA, -A, —NH₂, —NHA, —NA₂, —NH—(CH₂—CH₂—O)ₙ—H, —N[(CH₂—CH₂—O)ₙ—H]₂ or 2H-benzotriazol-2-y and/or
in formula XIV, W denotes CH₂—CH₂—O or CH₂—CH₂—CH₂—C(O)O and/or
in formula XV, R³ denotes —OH, —OA, -A, —NH₂, —NHA, —NA₂, —NH—(CH₂—CH₂—O)ₙ—H, —N[(CH₂—CH₂—O)ₙ—H]₂, —[NHA₂]X, —[NA₃]X, —SO₃H, —[SO₃]X, 2H-benzotriazol-2-yl or

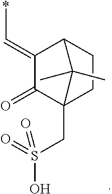 [SMILES: */C=C1/C(=O)C2(CS(=O)(=O)O)CCC1C2(C)C]

6. Process for the preparation of compounds according to claim 4, characterised in that a compound of the formula XVI 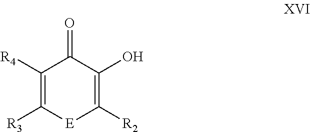
in which R₂, R₃, R₄ and E have a meaning indicated in claim 4,
is reacted with a compound of the formula XVII
R₁-M XVII,
in which R₁ has a meaning described in claim 4, where the part-formulae XI and XII are excluded, and
M denotes alkali metal or alkaline-earth metal cation or H or
a compound of the formula XVI, in which R₂, R₃, R₄ and E have a meaning indicated in claim 4,
is reacted with an acid halide or an active ester of the free acids derived from the formulae II, III, IV, V, VI, VII, XI, XII and XIV of claim 4.

7. Composition comprising at least one compound according to claim 4.

8. Composition according to claim 7, characterised in that it comprises a cosmetic, dermatological or pharmacologically tolerated vehicle.

9. Composition according to claim 7, characterised in that at least one further active compound is present selected from the group UV filters, antioxidants, vitamins, antiageing active compounds, anticellulite active compounds, self-tanning substances, antipigmentation substances or skin-lightening substances.

10. Composition comprising 2-methyl-4-oxo-4H-pyran-3-yl (E)-3-(4-hydroxy-phenyl)acrylate or 2-methyl-4-oxo-4H-pyran-3-yl 2-hydroxybenzoate, a cosmetic, dermatological or pharmacologically tolerated vehicle and at least one further active compound selected from the group UV filters, antioxidants, vitamins, antiageing active compounds, anticellulite active compounds, self-tanning substances, antipigmentation substances or skin-lightening substances.

11. Process for the preparation of a composition according to claim 7, characterised in that at least one compound of formula I is mixed with a vehicle and optionally with further active substances or assistants.

12. A functionalized protein-containing matrix comprising a structural unit of the formula I-1 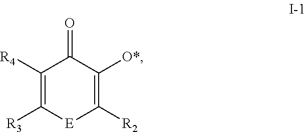
in which R₂, R₃ or R₄ each, independently of one another, denote —H, -A, —OA-, —(CH₂)ₚ—OH, —C(O)OA, COOH or COOX,
p denotes an integer from 1 to 4,
X is the counterion to the [COO⁻] group, and where
E denotes NR₅ or O,
as linker functionalizing the protein-containing matrix, where the symbol * denotes the linking site in the form of a covalent single bond to a molecule which is intended to be adsorbed onto or covalently bonded to the matrix.
